(19) United States
(12) Patent Application Publication
(10) Pub. No.: US 2026/0232644 A1
(43) Pub. Date: Aug. 13, 2026

(54) Compositions Comprising an Inhibitor of Galectin-3

(71) Applicant: University of Liverpool, Liverpool (GB)

(72) Inventor: Lu-Gang Yu, Liverpool (GB)

(73) Assignee: University of Liverpool, Liverpool (GB)

(21) Appl. No.: 19/495,682

(22) PCT Filed: Jul. 3, 2024
(86) PCT No.: PCT/GB2024/051726

§ 371 Date: Dec. 19, 2025

(30) Foreign Application Priority Data
Jul. 4, 2023 (GB) 2310252.8

(51) Int. Cl. A61K 31/4375; A61K 31/138; A61K 31/472; A61P 35/00

(52) U.S. Cl. CPC A61K 31/4375; A61K 31/138; A61K 31/472; A61P 35/00

(57) ABSTRACT

The present disclosure provides a composition comprising a compound of Formula X, or a pharmaceutically acceptable salt thereof, and/or a compound of Formula Y, or a pharmaceutically acceptable salt thereof, and/or a compound of Formula Z, or a pharmaceutically acceptable salt thereof, for use in inhibiting Galectin-3. The composition may be for use in the treatment and/or prevention of a disease, disorder, state or condition wherein inhibiting Galectin-3 would be beneficial for the treatment and/or prevention of the disease, disorder, state or condition. The disease, disorder, state or condition may be cancer, cancer metastasis, fibrosis, inflammation, diabetes and/or one or more other disease, disorder, state or condition affected and/or mediated by Galectin-3.

The present disclosure relates to the prevention and/or treatment of cancer and/or cancer metastasis and/or fibrosis and/or inflammation and/or diabetes and/or other conditions which are affected and/or mediated by Galectin-3. More specifically, the present disclosure relates to the prevention and/or treatment of cancer and/or cancer metastasis and/or fibrosis and/or inflammation and/or diabetes by inhibiting the activity of Galectin-3. The present disclosure relates to compounds and compositions for inhibiting the activity of Galectin-3. The present disclosure relates to methods and kits for inhibiting the activity of Galectin-3, particularly in methods of treatment for cancer and/or cancer metastasis and/or fibrosis and/or inflammation and/or diabetes and/or one or more other diseases, disorders, states or conditions which are affected and/or mediated by Galectin-3.

### BACKGROUND

In the UK cancer is a leading cause of death, accounting for one out of every four deaths with a total of more than 150,000 deaths each year. Nearly half of all people will develop cancer during their lifetime with more than 500,000 new cancer cases diagnosed each year. Despite enormous effort, effective treatment of this disease still remains a big challenge. It is increasingly believed that therapies targeted at specific molecules or mechanisms, especially multi-modal treatments hold the solution for more effective cancer treatments.

Cancer refers to a group of diseases which are characterised by abnormal and/or uncontrolled cell growth. These cells can form tumours, a tumour can develop when cells reproduce too quickly. The United States National Cancer Institute defines a tumour as “an abnormal mass of tissue that results when cells divide more than they should or do not die when they should.” Tumours can be any size, and can roughly be characterised as benign, premalignant or malignant. Malignant tumours can grow and spread to other parts of the body, for example via the blood or lymphatic systems. Benign tumours do not invade or destroy nearby tissues, or spread to other areas of the body. Premalignant tumours have not yet begun to spread, but may soon.

Galectin-3 (GAL3) is a family member of the galactoside-binding proteins and is expressed by many types of human cells, in particular epithelial and immune cells.

It is commonly overexpressed by most types of solid tumours such as breast, lung, colon, pancreatic, prostate, digestive or gastrointestinal tract, urinary system, thyroid and melanoma [Newlaczyl, A. U. and L. G. Yu, Galectin-3: A-jack-of-all-trades in cancer. Cancer Lett, 2011. 313: p. 123-128, Liu, F.-T. T. and G. A. Rabinovich, Galectins as modulators of tumour progression. Nature Reviews Cancer, 2005. 5(1): p. 29-41]. Extensive research over the past decades has revealed that Galectin-3 is a very important promoter of cancer progression and metastasis. It does so through multiple mechanisms intracellularly and extracellularly. Extracellular Galectin-3 interacts with galactoside-terminated glycans expressed on a number of cell surface molecules such as mucin proteins, growth factors and cell adhesion molecules (e.g. MUC1, MUC16, epidermal growth factor (EGF), transforming growth factor β (TGFβ) receptors, integrins) and basement matrix proteins that promote tumour cell adhesion, invasion and angiogenesis in cancer progression and metastasis. Extracellular Galectin-3 induces T cell apoptosis, prevents T-cell infiltration in the tumour microenvironment and dampens anti-tumour immunity in the body [Gordon-Alonso, M., et al., Galectin-3 captures interferon-gamma in the tumor matrix reducing chemokine gradient production and T-cell tumor infiltration. Nat Commun, 2017. 8(1): p. 793]. Galectin-3 is also known to promote tumour cell proliferation, establishment of metastatic niches, oncogenic signalling, apoptosis resistance and cell cycle progression when present intracellularly. Levels of circulating Galectin-3 are markedly elevated in cancer patients, in particular those with metastasis [Barrow, H., et al., Serum Galectin-2,-4, and -8 Are Greatly Increased in Colon and Breast Cancer Patients and Promote Cancer Cell Adhesion to Blood Vascular Endothelium. Clinical Cancer Research, 2011. 17(22): p. 7035-7046]. Circulating Galectin-3 has been found to have an important role in promoting circulating tumour cell metastatic spread to remote organs by interaction with disseminating tumour cells [Zhao, Q., et al., Interaction between circulating galectin-3 and cancer-associated MUC1 enhances tumour cell homotypic aggregation and prevents anoikis. Mol Cancer, 2010. 9: p. 154, Zhao, Q., et al., Circulating galectin-3 promotes metastasis by modifying MUC1 localization on cancer cell surface. Cancer Res, 2009. 69(17): p. 6799-806], as well as with the vascular endothelium [Chen, C., et al., Increased circulation of galectin-3 in cancer induces secretion of metastasis-promoting cytokines from blood vascular endothelium. Clinical Cancer Research 2013, 19:1693-704].

Pathological fibrosis results from abnormal tissue repair, from cellular stress, chronic, inflammation and/or severe tissue damage. It can lead to organ failure and is linked to chronic fibroproliferative diseases such as IPF24 and chronic inflammatory diseases such as NASH19. In addition to its identified role in cancer, Galectin-3 has recently been demonstrated to also be actively involved in tissue fibrosis (such as in the lung, liver, heart and kidney) and is critical in certain conditions of heart failure by promoting pro-inflammatory cytokine secretion, fibroblast proliferation/transformation and collagen production [Li, L. C., J. Li, and J. Gao, Functions of galectin-3 and its role in fibrotic diseases. J Pharmacol Exp Ther, 2014. 351(2): p. 336-43]. Galectin-3 expression is higher than normal in fibrotic lesions in humans. There are very limited drugs for fibrotic diseases, so identification of new therapies for fibrotic diseases is also underway. Given its links to tissue fibrosis, Galectin-3 is also a promising target in fibrotic disease research.

Due to its broad and profound impact on promotion of cancer progression, metastasis and tissue fibrosis, Galectin-3 is now considered an attractive and drug-able therapeutic target in these disease areas. Development of Galectin-3-targeted therapeutic drugs is underway. These include Small-molecule carbohydrate-conjugate inhibitors, polysaccharides, peptides and biological agents (e.g. siRNAs and neutralizing mAbs) and some carbohydrate-based compounds have shown promise in early phase clinical trials [Marino, K. V., et al., Targeting galectin-driven regulatory circuits in cancer and fibrosis. Nat Rev Drug Discov, 2023. 22(4): p. 295-316]. However, carbohydrates do not generally make good drugs due to their metabolism and poor physicochemical properties [Wang, J., et al., Exploring Carbohydrates for Therapeutics: A Review on Future Directions. Front Pharmacol, 2021. 12: p. 756724].

There still remains a need to provide compounds, preferably small molecular compounds, with favourable manufacture pathways and biosafety, providing inhibition of galectins, particularly Galectin-3.

Particularly, Galectin inhibitors which are known to only or preferentially target specific galectins would be advantageous. Several known Galectin inhibitors are known to bind to multiple galactoside-binding proteins (e.g. Galectin-1, Galectin-3 and Galectin-8). Targeting specific Galectin proteins could lead to a better understanding of the role of that specific Galectin in disease mechanisms, and better control of the effects of utilising such inhibitors in disease treatment and management.

In view of the foregoing, it is desirable to provide new therapeutic strategies for the treatment and prevention of cancers and of cancer metastasis, fibrosis and fibrotic diseases, diabetes and inflammation and inflammatory diseases, as well as other diseases, disorders, states or conditions affected and/or mediated by Galectin-3. It is an object of the present invention to provide compounds and compositions that are useful for the treatment of diseases, disorders, states and/or conditions which are affected and/or mediated by Galectin-3, such as cancer, diabetes, inflammation or fibrosis. Providing inhibitors which target specific Galectin family members is also desirable.

### SUMMARY OF THE DISCLOSURE

The present invention resides in the recognition that certain compounds (as defined hereinafter, compounds of Formula X, Formula Y and Formula Z) act as inhibitors of Galectin-3 activity. These compounds have been previously known, but never previously identified as inhibitors of Galectin-3 activity, and thus never identified as useful in targeting galectin-3 mediated diseases (e.g. in treatment of cancer, inflammation, fibrosis, diabetes etc.) identified herein. Accordingly, the compounds and compositions of the present invention are potentially useful agents for the prevention and/or treatment of diseases, disorders, states or conditions affected and/or mediated by Galectin-3, for example cancer and/or cancer metastasis and/or fibrosis and/or inflammation and/or diabetes. Further diseases, disorders, states or conditions affected and/or mediated by Galectin-3 include Asthma, Atherosclerosis, Atopic Dermatitis, Cerebral infarction, COPD, Degenerative Aortic Stenosis, Endometriosis, Encephalitis, Gastritis, HIV infection, Interstitial lung disease, Juvenile Idiopathic Arthritis, CVD mortality, Non-alcoholic steatohepatitis (NASH), Obesity, Pneumonia, Pulmonary hypertension, Plaque Psoriasis, Q Fever, Rheumatoid Arthritis, Systemic Sclerosis, Urinary tract infections, Covid-19, sepsis, Thrombosis, Venous Thrombosis, Wound Healing, Cardiac syndrome X (CSX), Yeast infection-Candidiasis and Zoster-related pain (allodynia). These may be referred to as diseases, disorders, states or conditions which are affected and/or mediated by Galectin-3 herein. The compounds identified herein may also be used for the prevention and/or treatment of any of these diseases, disorders, states or conditions. The compounds herein may be used for the prevention and/or treatment, or for use in the prevention and/or treatment, of any of these disorders, states or conditions affected and/or mediated by Galectin-3.

Herein the compounds, their metabolized and/or hydrolysed and/or oxidised forms, or pharmaceutically acceptable salts, hydrates or solvates thereof, or compositions of these, will act to inhibit Galectin-3, where inhibition of Galectin-3 will be beneficial to a subject, having use in the treatment and/or prevention of a disease, disorder, state or condition, for example wherein the disease, disorder, state or condition is cancer and/or cancer metastasis and/or fibrosis and/or inflammation and/or diabetes, and/or one or more other diseases, disorders, state or conditions affected and/or mediated by Galectin-3, the other diseases, disorders, states or conditions affected and/or mediated by Galectin-3 may be for example one or more of Asthma, Atherosclerosis, Atopic Dermatitis, Cerebral infarction, COPD, Degenerative Aortic Stenosis, Endometriosis, Encephalitis, Gastritis, HIV infection, Interstitial lung disease, Juvenile Idiopathic Arthritis, CVD mortality, Non-alcoholic steatohepatitis (NASH), Obesity, Pneumonia, Pulmonary hypertension, Plaque Psoriasis, Q Fever, Rheumatoid Arthritis, Systemic Sclerosis, Urinary tract infections, Covid-19, sepsis Thrombosis, Venous Thrombosis, Wound Healing, Cardiac syndrome X (CSX), Yeast infection-Candidiasis and Zoster-related pain (allodynia). Fibrosis hereinafter refers to fibrosis which occurs in any part of the body such as in liver, lung, heart, kidney. Inflammation hereinafter refers to inflammation which occurs from the stimulation of the body by internal or external inflammatory factors, or a combination or both, and the resulting effects, conditions, states, diseases and disorders. Methods and compositions for preventing and inhibiting cancer metastasis, cancer cell proliferation, cancer reappearance after initial therapeutic treatment or intervention, tumour growth, tumour angiogenesis or fibrosis are provided.

Compounds of and compositions comprising Formula X (Tosufloxacin tosylate) and compounds of and compositions comprising of Formula Y (Nelfinavir mesylate) and compounds of and compositions comprising of Formula Z (Toremifene citrate) have been shown for the first time to strongly inhibit Galectin-3 binding to its ligands. These compounds are known, but for the first time have been shown to inhibit Galectin-3 and thus affect Galectin-3-mediated tumour cell adhesion, invasion, angiogenesis in vitro. These have also been shown to inhibit Galectin-3-mediated secretion of inflammatory cytokines from macrophages which are fundamental in inflammation as well as in the process of fibrosis and formation of fibrosis. The compound of Formula X has shown little/no cytotoxicity and no detectable genotoxicity in yeast.

A compound of Formula W (Gemifloxacin) has also been identified as potential Galectin-3 inhibitor due to the structural similarity with the compound of Formula X. Gemifloxacin is also a fluoroquinolone with structural similarities with the compounds identified herein (particularly the compound of Formula X, Tosufloxacin tosylate). The compound of Formula W has been shown to impact pro-metastatic markers and because it has been shown to inhibit migration, invasion and metastasis by suppressing the activation of NFKB induced by TNFα in human breast cancer cells [T. C. Chen, Y. L. Hsu, Y. C. Tsai, Y. W. Chang, P. L. Kuo, Y. H. Chen, Gemifloxacin inhibits migration and invasion and induces mesenchymal-epithelial transition in human breast adenocarcinoma cells, J. Mol. Med., 92 (1) (2014), pp. 53-64; and Vikas Yadav a, Puneet Talwar, Repositioning of fluoroquinolones from antibiotic to anti-cancer agents: An underestimated truth, Biomedicine & Pharmacotherapy Volume 111, March 2019, Pages 934-946]. Gemifloxacin has also been shown to inhibit migration and invasion of human colon cancer cells [Kan J Y, Hsu Y L, Chen Y H, Chen T C, Wang J Y, Kuo P L. Gemifloxacin, a fluoroquinolone antimicrobial drug, inhibits migration and invasion of human colon cancer cells. Biomed Res Int. 2013; 2013:159786. Doi: 10.1155/2013/159786. Epub 2013 Dec. 10. PMID: 24386633; PMCID: PMC3872387.]. The effect of Gemifloxacin on the markers shown in breast cancer cells [NFKB(down), Snail(down), pI\ddot{k}B(down), I\acute{k}B(up), RKIP(up), N-Cad(down), Vimentin(down), E-Cad(up), SM-actin(down)] and colon cancer cells [NFKB(down), Snail(down), E-Cad(up), N-Cad(down), Claudin3 (up), Vimentin (down), pI\acute{k}B(down), I\acute{k}B(up), TAK1(down)] has led to the conclusion that the compound of Formula W (Gemifloxacin) is acting as a Galectin-3 inhibitor, because many of these markers (e.g. N-cad, vimentin, snail, E-cad, etc.) are all related to epithelial to mesenchymal transition (EMT, an important step in epithelial cancer development). Galectin-3 is known to be involved in EMT as well as in NFK-a signalling so it is likely Gemifloxacin effect reported in these citations was related to effect on Galectin-3.

The compounds identified herein have never before been identified as i) inhibitors of Galectin-3 or ii) potentially potent in the treatment and/or prevention of a disease, disorder, state or condition, for example wherein the disease, disorder, state or condition is cancer and/or cancer metastasis and/or tissue fibrosis and/or inflammation and/or diabetes or any other related disease, disorder, state or condition affected and/or mediated by Galectin-3.

These small molecular weight synthetic compounds have been identified as very potent Galectin-3 binding inhibitors. The compound of Formula X has been shown to bind to Galectin-3 with low micro molar affinity (full-length and C-terminal galectin-3 with binding affinity (KD) at 5~7 μM). The compounds of Formula X, Formula Y and Formula Z all strongly inhibit Galectin-3 binding to its ligand (IC50 all ~10 μM or below) and the compounds of Formula X and Formula Y have been proven to abolish various Galectin-3-mediated cancer cell activities such as angiogenesis, cell adhesion and migration. Efficacy has been proven with multiple cancer cell types, showing efficacy for use in treatment of multiple cancer types. The compounds of Formula X, Formula Y and Formula Z are also shown to inhibit Galectin-3-induced secretion of pro-inflammatory cytokines (TGFα and IL-1β) from macrophages, a clear indication of being able to affect inflammatory and fibrosis related diseases, disorders, state or condition. Secretion of pro-inflammatory cytokines is one of the fundamental mechanisms in Galectin-3-mediated pathogenesis of fibrosis, inhibition of this will inhibit or treat fibrosis. Moreover, the compound of Formula X shows no detectable cytotoxicity to either normal or cancerous human cells, and Compounds Y and Z show very low cytotoxicity to normal and cancer cells.

Furthermore, the compound of Formula X has been shown to not affect binding of Galectins-2, -4 and -8 to asialofetuin, thus the compound of Formula X has been identified as a selective inhibitor of Galectin-3. Inhibitors which selectively bind just one Galectin protein, e.g. Galectin-3, are particularly advantageous, as different galectin members can sometime have different influences on cancer activities.

These compounds have particularly been shown to have efficacy in multiple cancer types, including breast cancer (including triple negative breast cancer), pancreatic cancer, lung cancer, colorectal cancer and melanoma cells. The results from the Galectin-3 knockdown and knockout models indicate that the inhibition of Galectin-3 is key to the action of these compounds. Demonstrated herein is use of these compounds across a range of cancers.

“Inhibiting Galectin-3” as referred to herein refers to inhibition of Galectin-3 binding to its ligands and inhibition of Galectin-3-mediated activities of cells or tissues/organs, this may be related to diseases, disorders, states or conditions. The compounds described herein are known to bind to Galectin-3 protein, thus inhibiting Galectin-3-mediated actions and mechanisms in Galectin-3-related diseases, disorders, states or conditions.

A disease, disorder, state or condition where inhibiting Galectin-3 would be beneficial for the treatment and/or prevention of the disease, disorder, state or condition may be a disease, disorder, state or condition mediated by Galectin-3. The disease, disorder, state or condition mediated by Galectin-3 may be any of the diseases, disorders, state or condition listed in this application. Such disease, disorder, state or condition mediated by Galectin-3 may be related to any expression of Galectin-3 or overexpression of Galectin-3, or any known involvement of Galectin-3 in the disease, disorder, state or condition's progression, at any stage, including before the disease, disorder, state or condition has begun.

According to an aspect of the present invention, there is provided a compound of Formula X, or a pharmaceutically acceptable salt thereof, or a composition thereof, for use as a medicament. According to an aspect of the present invention, there is provided a compound of Formula Y, or a pharmaceutically acceptable salt thereof, or a composition thereof, for use as a medicament. According to an aspect of the present invention, there is provided a compound of Formula Z, or a pharmaceutically acceptable salt thereof, or a composition thereof, for use as a medicament.

According to an aspect of the present invention, there is provided a compound of Formula X, or a pharmaceutically acceptable salt thereof, or a composition thereof, for use in the treatment and/or prevention of a disease, disorder, state or condition. According to an aspect of the present invention, there is provided a compound of Formula Y, or a pharmaceutically acceptable salt thereof, or a composition thereof, for use in the treatment and/or prevention of a disease, disorder, state or condition. According to an aspect of the present invention, there is provided a compound of Formula Z, or a pharmaceutically acceptable salt thereof, or a composition thereof, for use in the treatment and/or prevention of a disease, disorder, state or condition.

According to an aspect of the present invention, there is provided a compound of Formula X, or a pharmaceutically acceptable salt thereof, or a composition thereof, for use in the treatment and/or prevention of cancer, cancer metastasis, fibrosis, inflammation or diabetes. According to an aspect of the present invention, there is provided a compound of Formula Y, or a pharmaceutically acceptable salt thereof, or a composition thereof, for use in the treatment and/or prevention of cancer, cancer metastasis, fibrosis, inflammation or diabetes. According to an aspect of the present invention, there is provided a compound of Formula Z, or a pharmaceutically acceptable salt thereof, or a composition thereof, for use in the treatment and/or prevention of cancer, cancer metastasis, fibrosis, inflammation or diabetes.

According to an aspect of the present invention, there is provided a compound of Formula X, or a pharmaceutically acceptable salt thereof, or a compound of Formula Y, or a pharmaceutically acceptable salt thereof, or a compound of Formula Z, or a pharmaceutically acceptable salt thereof, for use in inhibiting or inhibition of Galectin-3, preferably for use in the treatment and/or prevention of a disease, disorder, state or condition where inhibiting Galectin-3 would be beneficial for the treatment and/or prevention of the disease, disorder, state or condition. Also provided herein is the compound of Formula W for use in inhibiting or inhibition of Galectin-3, preferably for use in the treatment and/or prevention of a disease, disorder, state or condition where inhibiting Galectin-3 would be beneficial for the treatment and/or prevention of the disease, disorder, state or condition.

According to a further aspect of the present invention, there is provided a composition comprising a compound of Formula X, and/or a pharmaceutically acceptable salt thereof, and/or a compound of Formula Y, or a pharmaceutically acceptable salt thereof, and/or a compound of Formula Z, or a pharmaceutically acceptable salt thereof, or a combination of these, for use in inhibiting Galectin-3, preferably for use in the treatment and/or prevention of a disease, disorder, state or condition where inhibiting Galectin-3 would be beneficial for the treatment and/or prevention of the disease, disorder, state or condition.

According to a further aspect of the present invention, there is provided a composition comprising a compound of Formula X, or a pharmaceutically acceptable salt thereof, and/or a compound of Formula Y, or a pharmaceutically acceptable salt thereof, and/or a compound of Formula Z, or a pharmaceutically acceptable salt thereof, or a combination of these, for use in the treatment and/or prevention of a disease, disorder, state or condition. Preferably the disease, disorder, state or condition is mediated by Galectin-3. Preferably, the disease, disorder, state or condition is cancer, cancer metastasis, fibrosis, inflammation or diabetes. Preferably, the disease, disorder, state or condition is cancer, cancer metastasis, fibrosis, inflammation or diabetes and/or one or more other disease, disorder, state or condition affected and/or mediated by Galectin-3.

According to an aspect of the present invention, there is provided a compound of Formula X, or the metabolized and/or hydrolysed and/or oxidised form, or a pharmaceutically acceptable salt thereof, for use in inhibiting Galectin-3 in vitro or in vivo. According to an aspect of the present invention, there is provided a compound of Formula Y, or the metabolized and/or hydrolysed and/or oxidised form, or a pharmaceutically acceptable salt thereof, for use in inhibiting Galectin-3 in vitro or in vivo. According to an aspect of the present invention, there is provided a compound of Formula Z, or the metabolized and/or hydrolysed and/or oxidised form, or a pharmaceutically acceptable salt thereof, for use in inhibiting Galectin-3 in vitro or in vivo. According to a further aspect of the present invention, there is provided a compound of Formula X, and/or a pharmaceutically acceptable salt thereof, and/or a compound of Formula Y, or a pharmaceutically acceptable salt thereof, and/or a compound of Formula Z, or a pharmaceutically acceptable salt thereof, or a combination of these, for use in inhibiting Galectin-3 in vitro or in vivo.

The use according to any aspect of the invention herein may comprise administering an effective amount of the composition comprising or the compound of Formula X and/or the compound of Formula Y and/or the compound of Formula Z, and/or the compound of Formula W, or the metabolized and/or hydrolysed and/or oxidised form or the pharmaceutically acceptable salt of those compounds, or a composition of one or more of these compounds.

According to a further aspect of the present invention, there is provided a compound of Formula X, or a pharmaceutically acceptable salt thereof, and/or the compound of Formula Y and/or the compound of Formula Z, for use in the treatment and/or prevention of cancer, cancer metastasis, diabetes, fibrosis, inflammation and/or one or more other disease, disorder, state or condition affected and/or mediated by Galectin-3.

According to a further aspect of the present invention, there is provided a composition comprising a compound of Formula X, or a pharmaceutically acceptable salt thereof, and/or the compound of Formula Y, or a pharmaceutically acceptable salt thereof, and/or the compound of Formula Z, or a pharmaceutically acceptable salt thereof, for use in the treatment and/or prevention of cancer, cancer metastasis, fibrosis, inflammation and/or diabetes and/or one or more other disease, disorder, state or condition affected and/or mediated by Galectin-3. Preferably, the compound of Formula X, or a pharmaceutically acceptable salt thereof, and/or the compound of Formula Y, or a pharmaceutically acceptable salt thereof, and/or the compound of Formula Z, or a pharmaceutically acceptable salt thereof, may be in an admixture with a pharmaceutically acceptable diluent or carrier, which may be in a composition.

According to a further aspect of the present invention, there is provided a method of treating and/or preventing a disease, disorder, state or condition, the method comprising administering to the subject a composition, the composition comprising a compound of Formula X, or a pharmaceutically acceptable salt thereof and/or a compound of Formula Y, or a pharmaceutically acceptable salt thereof, and/or a compound of Formula Z, or a pharmaceutically acceptable salt thereof. Preferably, the disease, disorder, state or condition is cancer, cancer metastasis, fibrosis, inflammation and/or diabetes. Preferably, the disease, disorder, state or condition is mediated by Galectin-3.

According to a further aspect of the present invention, there is provided a method of prevention, management, amelioration and/or treatment of a disease, disorder, state or condition, preferably wherein the a disease, disorder, state or condition, is cancer, cancer metastasis, fibrosis, inflammation and/or diabetes, the method comprising administering a therapeutically effective amount of a composition to a subject in need thereof, the composition comprising a compound of Formula X, or a pharmaceutically acceptable salt thereof and/or a compound of Formula Y, or a pharmaceutically acceptable salt thereof, and/or a compound of Formula Z, or a pharmaceutically acceptable salt thereof, and/or the method comprising administering one or more compositions comprising Formula X and/or Formula Y and/or Formula Z, or pharmaceutically acceptable salts thereof.

According to a further aspect of the present invention, there is provided a method of treating and/or preventing a disease, disorder, state or condition, wherein inhibiting Galectin-3 would be beneficial for the treatment of the disease, disorder, state or condition, or wherein the disease, disorder, state or condition is affected or mediated by Galectin-3, the method comprising administering to the subject a composition, the composition comprising the compound of Formula X, or a pharmaceutically acceptable salt thereof, and/or the compound of Formula Y, or a pharmaceutically acceptable salt thereof, and/or the compound of Formula Z, or a pharmaceutically acceptable salt thereof. Preferably, the disease, disorder, state or condition is mediated by Galectin-3.

According to a further aspect of the present invention, there is provided a method of prevention, management, amelioration and/or treatment of cancer, cancer metastasis, fibrosis, inflammation and/or diabetes and/or one or more other disease, disorder, state or condition affected and/or mediated by Galectin-3, the method comprising administering a therapeutically effective amount of a composition to a subject in need thereof, the composition comprising the compound of Formula X, or a pharmaceutically acceptable salt thereof, and/or the compound of Formula Y, or a pharmaceutically acceptable salt thereof, and/or the compound of Formula Z, or a pharmaceutically acceptable salt thereof, and/or the method comprising administering more than one composition comprising Formula X and/or Formula Y and/or Formula Z, or pharmaceutically acceptable salts thereof. Preferably, inhibiting Galectin-3 would be beneficial for the treatment and/or prevention of the disease, disorder, state or condition. Administration of one or more compounds or compositions may be in any order or regime as described herein.

In a related aspect, the invention may comprise the compound of Formula X, or a pharmaceutically acceptable salt thereof, and/or the compound of Formula Y, or a pharmaceutically acceptable salt thereof, and/or the compound of Formula Z, or a pharmaceutically acceptable salt thereof, for use in the manufacture of a medicament. Preferably the medicament is for use in cancer, cancer metastasis, fibrosis, inflammation, diabetes and/or one or more other disease, disorder, state or condition affected and/or mediated by Galectin-3, and/or in the manufacture of a medicament for use in inhibiting Galectin-3.

Also provided herein is the compound of Formula W for use in inhibiting or inhibition of Galectin-3, preferably for use in the treatment and/or prevention of a disease, or disorder, state or condition where inhibiting Galectin-3 would be beneficial for the treatment and/or prevention of the disease, or disorder, state or condition. According to a further aspect of the present invention, there is provided a composition comprising a compound of Formula X, and/or a pharmaceutically acceptable salt thereof, for use in inhibiting Galectin-3, preferably for use in the treatment and/or prevention of a disease, disorder, state or condition where inhibiting Galectin-3 would be beneficial for the treatment and/or prevention of the disease, disorder, state or condition. According to a further aspect of the present invention, there is provided a composition comprising a compound of Formula W. or a pharmaceutically acceptable salt thereof, or a combination of these, for use in the treatment and/or prevention of a disease, disorder, state or condition mediated by Galectin-3. Preferably, the disease, disorder, state or condition is cancer, cancer metastasis, fibrosis, inflammation or diabetes and/or one or more other disease, disorder, state or condition affected and/or mediated by Galectin-3. According to an aspect of the present invention, there is provided a compound of Formula W, or the metabolized and/or hydrolysed and/or oxidised form, or a pharmaceutically acceptable salt thereof, for use in inhibiting Galectin-3 in vitro or in vivo. According to a further aspect of the present invention, there is provided a compound of Formula W, or a pharmaceutically acceptable salt thereof for use in the treatment and/or prevention of cancer, cancer metastasis, diabetes, fibrosis, inflammation and/or one or more other disease, disorder, state or condition affected and/or mediated by Galectin-3. According to a further aspect of the present invention, there is provided a composition comprising a compound of Formula W, or a pharmaceutically acceptable salt thereof, for use in the treatment and/or prevention of cancer, cancer metastasis, fibrosis, inflammation and/or diabetes and/or one or more other disease, disorder, state or condition affected and/or mediated by Galectin-3. Preferably, the compound of Formula W, or a pharmaceutically acceptable salt thereof, may be in an admixture with a pharmaceutically acceptable diluent or carrier, which may be in a composition. According to a further aspect of the present invention, there is provided a method of treating and/or preventing a disease, disorder, state or condition, wherein inhibiting Galectin-3 would be beneficial for the treatment of the disease, disorder, state or condition, or wherein the disease, disorder, state or condition is affected or mediated by Galectin-3, the method comprising administering to the subject a composition, the composition comprising the compound of Formula W, or a pharmaceutically acceptable salt thereof. According to a further aspect of the present invention, there is provided a method of prevention, management, amelioration and/or treatment of cancer, cancer metastasis, fibrosis, inflammation and/or diabetes and/or one or more other disease, disorder, state or condition affected and/or mediated by Galectin-3, the method comprising administering a therapeutically effective amount of a composition to a subject in need thereof, the composition comprising the compound of Formula W, or a pharmaceutically a acceptable salt thereof. In a related aspect, the invention may comprise the compound of Formula W, or a pharmaceutically acceptable salt thereof, for use in the manufacture of a medicament for use in cancer, cancer metastasis, fibrosis, inflammation, diabetes and/or one or more other disease, disorder, state or condition affected and/or mediated by Galectin-3, and/or in the manufacture of a medicament for use in inhibiting Galectin-3.

The compounds and compositions of the previously described aspects may be for use in the manufacture of a medicament for the prevention and/or treatment of a disease, disorder, state or condition described herein, preferably for use in the manufacture of a medicament for cancer, cancer metastasis, fibrosis, inflammation, diabetes, and/or one or more other disease, disorder, state or condition affected and/or mediated by Galectin-3, and/or for use in the manufacture of a medicament for use in inhibiting Galectin-3.

Preferably, for all aspects, the composition is a pharmaceutical composition comprising a compound of Formula X and/or Formula Y and/or Formula Z, and/or Formula W, or a pharmaceutically acceptable salt thereof, and a pharmaceutically acceptable excipient. The compound of Formula X and/or Formula Y and/or Formula Z and/or Formula W may be in a metabolized and/or hydrolysed and/or oxidised form, or a pharmaceutically acceptable salt, hydrate or solvate thereof.

Preferably, the disease, disorder, state or condition is mediated by Galectin-3. Preferably, the disease, disorder, state or condition is cancer, cancer metastasis, fibrosis, inflammation, diabetes, and/or a disease, disorder, state or condition associated with or related to those, and/or one or more other disease, disorder state or condition affected and/or mediated by Galectin-3.

Preferably, the treatment and/or prevention of cancer comprises inhibiting metastasis, inhibiting cancer reappearance after initial therapeutic treatment or intervention, inhibiting cancer cell proliferation, inhibiting tumour growth, inhibiting angiogenesis or inhibiting immunosuppression.

Preferably, the cancer is one or more cancers selected from the group consisting of breast cancer, including triple negative breast cancer, pancreatic cancer, colorectal cancer, head and neck cancer, ovarian cancer, lung cancer, melanoma, thyroid cancer, bladder cancer, urinary system cancer, oesophageal cancer, prostate cancer, digestive or gastrointestinal tract cancer, leukaemia, lymphoma, liver cancer and musculoskeletal cancer. Preferably, the cancer is breast cancer, pancreatic cancer, colorectal cancer, lung cancer, pancreatic cancer, or melanoma. Preferably, the breast cancer is triple negative breast cancer. Preferably the cancer is colorectal cancer. Preferably the cancer is pancreatic cancer. Preferably the cancer is melanoma. Preferably the cancer is lung cancer. Preferably, the cancer is triple negative breast cancer.

Preferably, the treatment and/or prevention of cancer metastasis comprises inhibition of the motility of cancer or tumour cells, inhibition of angiogenesis, inhibition of immunosuppression, inhibition of the dissemination and invasiveness of cancer cells leading to inhibition of metastatic tumour growth, inhibitor of the dissemination and invasiveness of cancer cells leading to improved clinical outcome for the subject.

Preferably, the treatment and/or prevention of fibrosis comprises treatment/prevention of a fibrotic disorder, a fibrotic condition, a fibroproliferative disease, or a disorder or state of wound-healing response that is out of control.

Preferably, fibrosis comprises inhibition of a fibrotic disease or a condition caused by fibrosis. Preferably, this may be scarring or wound healing abnormalities, chronic or acute inflammation, chronic graft rejection, or a fibrotic condition or fibrosis affecting the heart, brain, lungs, liver, kidneys, heart or vascular system, mediastinum, bone, retroperitoneum, skin, digestive or gastrointestinal tract, connective tissue, eye or muscles.

Preferably, the treatment and/or prevention of inflammation comprises treatment/prevention of an inflammatory disease or a condition caused by inflammation.

Preferably, inflammation comprises inhibition of an inflammation-related disease, disorder, state or condition caused by inflammation. Preferably, this may be an inflammation related disease or disorder such as, or such as one caused by, acute or chronic organ transplant rejection, graft-versus-host disease, inflammatory bowel disease, inflammatory skin disease, multiple sclerosis, arteriosclerosis, pancreatitis, acute bronchitis, chronic bronchitis, Alzheimer's disease, inflammatory lung disease, inflammatory skin disease, Acute bronchiolitis, folliculitis, chronic bronchiolitis, musculoskeletal pain or connective tissue inflammation, osteoarthritis, gout, spondyloarthropathy, Reiter's syndrome, psoriatic arthropathy, inflammation caused by bacterial, fungal, and viral infections one or more diseases, atherosclerosis, dermatitis, diverticulitis, fibromyalgia, hepatitis, irritable bowel syndrome, nephritis, Parkinson's disease, chronic inflammation, one or more neurodegenerative diseases or disorders caused by tumours caused by chronic inflammation and aging caused by chronic inflammation, inflammatory symptoms caused by autoimmune diseases including ulcerative colitis (UC) and Crohn's disease (CD), lupus erythematosus, hyperthyroidism, IgA nephritis, type I or type II diabetes and its complications, dry eye syndrome, rheumatoid arthritis, simple obesity, ankylosing spine inflammation, bronchial asthma, neurodermatitis, ulcerative colitis, oral ulcers, psoriasis, vitiligo, Behçet's disease, autoimmune iridocyclitis, autoimmune eczema, autoimmune uveitis, One or more of autoimmune conjunctivitis, autoimmune dry eye, autoimmune glaucoma, autoimmune cataract, allergic rhinitis, irritable bowel syndrome, pruritus.

Preferably, diabetes comprises type I or type II diabetes and/or any related diabetic-associated disease, disorder, state or condition, such as obesity.

Preferably, the one or more other disease, disorder, state or condition which is affected and/or mediated by Galectin-3 is Asthma, Atherosclerosis, Atopic Dermatitis, Cerebral infarction, COPD, Degenerative Aortic Stenosis, Endometriosis, Encephalitis, Gastritis, HIV infection, Interstitial lung disease, Juvenile Idiopathic Arthritis, CVD mortality, Non-alcoholic steatohepatitis (NASH), Obesity, Pneumonia, Pulmonary hypertension, Plaque Psoriasis, Q Fever, Rheumatoid Arthritis, Systemic Sclerosis, Urinary tract infections, Covid-19, sepsis, Thrombosis, Venous Thrombosis, Wound Healing, Cardiac syndrome X (CSX), Yeast infection-Candidiasis or Zoster-related pain (allodynia).

In all aspects, the compound of Formula X and/or Formula Y and/or Formula Z and/or Formula W may be a mimetic, or derivative thereof.

In all aspects, the compound of Formula X and/or Formula Y and/or Formula Z and/or Formula W may be in a metabolized and/or hydrolysed and/or oxidised form, or a pharmaceutically acceptable salt, hydrate or solvate thereof.

Preferably, the disease, disorder, state or condition of any aspect of the present invention, for example cancer, may be associated with expression of Galectin-3 or suspected of being associated with expression of Galectin-3.

Preferably, the methods described herein may also comprise first determining the level of Galectin-3 or expression of LGALS3 in a biological sample take from a subject. This may be prior to, sequentially and/or after with the herein described treatments with Galectin-3 inhibitors. This may include determining said level of or expression in a biological sample, for example a fluid (e.g. blood, tear or urine) sample or a tissue sample (e.g. a tissue biopsy, biopsy, tumour sample, suspected tumour sample) take from a subject. The Galectin-3 inhibitors may be one or more of the compounds Formula X and/or Formula Y and/or Formula Z, and/or Formula W, or a composition comprising one or more of these, or a pharmaceutically acceptable salt thereof.

Preferably, said subject may be suspected of having a disease, disorder, state or condition associated with expression of Galectin-3. Preferably, said subject may be known to have a disease, disorder, state or condition associated with expression of Galectin-3.

Preferably, biological sample or sample may be tumour tissue, suspected tumour tissue, a tissue biopsy, mucus, stool, tear, urine, blood, serum, cell extract, biopsy specimens and/or fluid which has been introduced into the body of an individual and subsequently removed.

Preferably, any method described herein may comprise determining the level of Galectin-3 or expression of LGALS3 or level of LGALS3 expression in a subject, for example in a biological sample taken from said subject. Preferably this is prior to a subject being given a Galectin-3 inhibitor such as a compound or composition comprising a compound of Formula X, and/or Formula Y and/or Formula Z and/or Formula W. This may be before any administration and the method may then comprise administering a composition comprising the compound of Formula X, or a pharmaceutically acceptable salt thereof, and/or a composition comprising the compound of Formula Y, or a pharmaceutically acceptable salt thereof, and/or a composition comprising the compound of Formula Z, or a pharmaceutically acceptable salt thereof, and/or a composition comprising the compound of Formula W, or a pharmaceutically acceptable salt thereof, optionally with a pharmaceutically acceptable excipient, to said subject, or any other such compounds, composition or administration as described herein. This administration may be carried out when any Galectin-3 or any expression of LGALS3 in the biological sample from said subject is found or determined. This may be administration may be carried out when the level of Galectin-3 or expression of LGALS3 or level of LGALS3 expression in the biological sample from said subject is found to be higher than a base line, higher than a reference, or overexpressed. Higher than a base line or overexpressed may be compared to a reference, for example a literature reference or a reference sample, as described herein. The reference/base line may be zero. The method may further comprise the step of administering a composition comprising the compound of Formula X, or a pharmaceutically acceptable salt thereof, and/or a composition comprising the compound of Formula Y, or a pharmaceutically acceptable salt thereof, and/or a composition comprising the compound of Formula Z, or a pharmaceutically acceptable salt thereof, and/or a composition comprising the compound of Formula W, or a pharmaceutically acceptable salt thereof, optionally with a pharmaceutically acceptable excipient, to said subject when the level of Galectin-3 or expression of LGALS3 or level of LGALS3 expression in the biological sample from said subject is found to be higher than a base line or overexpressed, for example greater than a reference, for example a literature reference or a reference sample. The reference/base line may be zero.

Accordingly, any method of treatment of the present invention may first involve diagnosing the subject as having a disease, disorder, state or condition associated with or suspected of being Galectin-3 or expression of LGALS3 or a level of LGALS3 expression greater than expected. This may be before administration.

According to a further aspect of the present invention, there is provided a method of inhibiting Galectin-3 in a patient or in a biological sample comprising the step of administering to said patient or contacting said biological sample with a compound of Formula X, or a pharmaceutically acceptable salt thereof, and/or a compound of Formula Y, or a pharmaceutically acceptable salt thereof, and/or a compound of Formula Z, or a pharmaceutically acceptable salt thereof, and/or a compound of Formula W, or a pharmaceutically acceptable salt thereof, or a composition comprising one or more of these compounds. Preferably, this may be in vitro.

Preferably, for all aspects, the compounds and/or compositions may be administered alone or in combination with other treatments, either simultaneously or sequentially. The other treatment may be chemotherapy, radiotherapy, immunotherapy and/or surgery.

Preferably, for all aspects, compositions according to the invention may further comprise other active agents, for example one or more additional anti-tumour agent and/or anti-metastatic agent and/or chemotherapy agent, and/or radiotherapy agent, and/or immunotherapeutic agent, and/or anti-fibrosis/fibrotic agents and/or anti-inflammation/inflammatory agents and/or one or more other Galectin inhibitor.

Preferably, the compounds and/or compositions may be administered in combination with at least one additional anti-tumour agents, anti-metastatic agents, chemotherapy agents, immunotherapeutic agents, surgery and/or radiotherapy agents, anti-fibrosis/fibrotic agents, anti-inflammation/inflammatory agents and/or one or more other Galectin inhibitors. The compounds and/or compositions may be administered in combination with surgical approaches to cancer, fibrosis or other disease treatments, for example tumour de-baulking or cryosurgery.

According to a further aspect of the present invention there is provided a kit consisting of separate packs, packages or amounts of an effective amount of one or more of: a compound of Formula X, a compound of Formula Y, a compound of Formula Z, a composition comprising the compound of Formula X, a composition comprising Formula Y, a composition comprising the compound of Formula Z, the compound of Formula W, and/or a composition comprising one or more of the compounds of Formula X and/or Formula Y and/or Formula Z and/or Formula W, and/or pharmaceutically acceptable salts or pharmaceutical formulations of any these, optionally including one or more pharmaceutically or cosmetically acceptable ingredients or excipients or separate packs, packages or amounts of these pharmaceutically or cosmetically acceptable ingredients or excipients, and/or optionally an effective amount of a further active ingredient such as those described herein. The kit may also optionally comprise suitable containers, such as boxes, individual bottles, bags or ampoules. The kit may, for example, comprise separate ampoules, each containing an effective amount of the compounds or compositions and/or pharmaceutically acceptable salts thereof, and optionally an effective amount of a further active ingredient, one or more of in dissolved or lyophilized form(s). The kit may further comprise instructions or means to administer one or more of the compounds or compositions of the kit. The kit may further comprise instructions to use the components in the treatment and/or prevention of a disease, disorder, state or condition where inhibiting Galectin-3 would be beneficial for the treatment and/or prevention of the disease, disorder, state or condition.

The compositions according to all aspects of the invention may further comprise one or more pharmaceutically or cosmetically acceptable ingredients or excipients. Pharmaceutically acceptable ingredients are well known to those skilled in the art, and include, but are not limited to, pharmaceutically acceptable carriers, adjuvants, excipients, diluents, fillers, buffers, preservatives, anti-oxidants, lubricants, stabilisers, solubilisers, surfactants (e.g. wetting agents), masking agents, colouring agents, fragrance agents and penetration agents.

Preferably, for all aspects, the compositions may be provided as a suspension in a pharmaceutically or cosmetically acceptable excipient, diluent or carrier.

Compounds and/or pharmaceutically acceptable salts thereof, thereof, or compositions of the present invention may be administered orally, parenterally, by inhalation spray, topically, rectally, nasally, buccally, vaginally or via an implanted reservoir. Preferably, they are administered orally, intraperitoneally or intravenously. Preferably, pharmaceutically acceptable compositions of this invention are formulated for oral administration. Exemplary oral dosage forms are capsules, tablets, aqueous suspensions or solutions. Such compositions are prepared according to techniques well-known in the art of pharmaceutical formulation.

The compositions of the present invention may be formulated as medicaments, that is to say formulated as a medicine, or a medical device. The medicament may include other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g. wetting agents), masking agents, colouring agents, flavouring agents, and sweetening agents. The Formulation may further comprise other active agents, for example other therapeutic or prophylactic agents.

The compounds and compositions according to the method of the present invention can be administered using any amount and any route of administration effective for treating or lessening the severity of a disorder provided above. The exact amount required will vary from subject to subject, depending on the species, age, and general condition of the subject, the severity of the disease, disorder condition or state, the particular agent, its mode of administration, and the like. This may comprise administration of an effective amount or a therapeutically effective amount of any of the compounds or compositions described herein.

Preferably, for all aspects, the compounds and/or compositions may be administered alone or in combination with other treatments, either simultaneously or sequentially. The other treatment may be chemotherapy, radiotherapy, immunotherapy and/or surgery. Compositions according to the invention may further comprise other active agents, for example one or more additional anti-tumour agents, anti-metastatic agents, chemotherapy agents, and/or radiotherapy agents and/or immunotherapeutic agents, and/or anti-fibrosis/fibrotic agents and/or anti-inflammation/inflammatory agents and/or one or more other Galectin inhibitors. The compounds and/or compositions may be administered in combination with at least one additional anti-tumour agents, anti-metastatic agents, chemotherapy agents, immunotherapeutic agents, surgery anti-fibrosis/fibrotic agents, anti-inflammation/inflammatory agents, one or more other Galectin inhibitor and/or radiotherapy agents. The compounds and/or compositions may be administered in combination with surgical approaches to cancer, fibrosis or other disease treatments, for example tumour de-baulking or cryosurgery.

According to a further aspect of the present invention, there is provided a combination suitable for use in the treatment of a cancer, fibrosis, inflammation, diabetes, cancer metastasis, and/or one or more other disease, disorder, state or condition affected and/or mediated by Galectin-3, the combination comprising a compound or composition of the invention as defined herein, or a pharmaceutically acceptable salt thereof, and another anti-tumour agent and/or anti-metastatic agent and/or chemotherapy agent and/or radiotherapy agent and/or immunotherapeutic agent, and/or anti-fibrosis/fibrotic agents and/or anti-inflammation/inflammatory agents and/or one or more other Galectin inhibitor.

In a further aspect of the invention there is provided a compound or composition of the invention as defined herein, or a pharmaceutically acceptable salt thereof, for use in the treatment of cancer, fibrosis, inflammation, diabetes, cancer metastasis, and/or one or more other disease, disorder, state or condition affected and/or mediated by Galectin-3, wherein the compound or composition is administered in combination with an anti-tumour agent and/or anti-metastatic agent and/or chemotherapy agent, and/or radiotherapy agent and/or immunotherapeutic agent, and/or anti-fibrosis/fibrotic agents and/or anti-inflammation/inflammatory agents and/or one or more other Galectin inhibitor.

According to a further aspect of the invention there is provided a pharmaceutical composition which comprises a compound of Formula X and/or a compound of Formula Y, and/or a compound of Formula Z, or a pharmaceutically acceptable salt thereof, and/or a compound of Formula W, or a pharmaceutically acceptable salt thereof, in combination with an anti-tumour agent and/or anti-metastatic agent and/or chemotherapy agent, and/or radiotherapy agent and/or immunotherapeutic agent and/or anti-fibrosis/fibrotic agent and/or anti-inflammation/inflammatory agent and/or one or more other Galectin inhibitor. Preferably, the pharmaceutical composition is for use in for use in inhibiting Galectin-3, preferably wherein the composition is for use in the treatment and/or prevention of a disease, disorder, state or condition where inhibiting Galectin-3 would be beneficial for the treatment and/or prevention of the disease, disorder, state or condition, or wherein the disease, disorder, state or condition is mediated by Galectin-3, preferably wherein the disease, disorder, state or condition is cancer, cancer metastasis, fibrosis, inflammation, diabetes and/or one or more other disease, disorder, state or condition affected and/or mediated by Galectin-3.

According to a further aspect of the invention, there is provided a method of treatment of a human or animal subject suffering from a cancer, fibrosis, inflammation, diabetes, cancer metastasis and/or one or more other disease, disorder, state or condition affected and/or mediated by Galectin-3, comprising administering to the subject a therapeutically effective amount of compound or composition of the invention as defined herein, or a pharmaceutically acceptable salt thereof, simultaneously, sequentially or separately with surgery, and/or at least one additional anti-tumour agent and/or anti-metastatic agent and/or chemotherapy agent, and/or radiotherapy agent, and/or immunotherapeutic agent, and/or anti-fibrosis/fibrotic agents and/or anti-inflammation/inflammatory agents and/or one or more other Galectin inhibitor as defined herein.

According to a further aspect of the invention, there is provided a compound or composition of the invention as defined herein, or a pharmaceutically acceptable salt thereof, for use simultaneously, sequentially or separately with surgery, and/or at least one additional anti-tumour agent and/or anti-metastatic agent and/or chemotherapy agent, and/or radiotherapy agent and/or immunotherapeutic agent and/or anti-fibrosis/fibrotic agent and/or anti-inflammation/inflammatory agent and/or one or more other Galectin inhibitor as defined herein, in the treatment of a cancer, fibrosis, inflammation, diabetes and/or cancer metastasis.

It will be appreciated that preferred features ascribed to one aspect of the invention applies mutatis mutandis to each and every aspect of the invention.

Though the present invention may relate to any compound or particular group of compounds for use in the methods defined herein by way of optional, preferred or suitable features or otherwise in terms of particular embodiments, the present invention may also relate to any compound or particular group of compounds that specifically excludes said optional, preferred or suitable features or particular embodiments.

### BRIEF DESCRIPTION OF THE DRAWINGS

Embodiments of the present disclosure will now be described with reference to the accompanying drawings, by way of example only, in which:

FIG. 1A, FIG. 1B and FIG. 1C show that compounds of Formula X, Formula Y and Formula Z inhibit Galectin-3 binding to its ligand asialofetuin (ASF). Binding of Galectin-3 (5 μg/ml) to asialofetuin (ASF, 20 μg/ml) in the absence or presence of different concentrations of the compound of Formula X (FIG. 1A), the compound of Formula Y (FIG. 1B) or the compound of Formula Z (FIG. 1C) was assessed by ELISA. Compounds X, Y and Z inhibit galectin-3 binding to ASF in a dose-dependent manner.

FIG. 2A, FIG. 2B, FIG. 2C and FIG. 2D show the analysis of the binding affinity of the compounds of Formula X, Formula Y and Formula Z to Galectin-3. The Binding of the compound of Formula X to full-length (FIG. 2A) and C-terminal (aa 115-250) Galectin-3 (FIG. 2B) is shown, along with the binding of Formula Y (FIG. 2C) and Formula Z (FIG. 2D) to Galectin-3. These were assessed by Tryptophan Fluorescence Spectroscopy (TFS). The compound of Formula X binds to the full-length and C-terminal galectin-3 with binding affinity (KD) at 5~7 μM. The compound of Formula Y binds to the full-length galectin-3 with binding affinity (KD) at ~90 μM. The compound of Formula Z binds to the full-length galectin-3 with binding affinity (KD) at ~35 μM.

FIG. 3A and FIG. 3B show that the compound of Formula X inhibits cancer cell adhesion to vascular endothelial cells. Adhesion of human melanoma ACA19 (FIG. 3A) and colon cancer SW620 (FIG. 3B) cells to human umbilical vein endothelial cells (HUVECs) was assessed in the absence or presence of Galectin-3 (2 μg/ml) and various concentrations of the compound of Formula X. The compound of Formula X causes dose-dependent inhibition of SW620 and ACA19 cell adhesion to HUVECs. ***P<0.001, **p<0.01, *p<0.05 (ANOVA)

FIG. 4A and FIG. 4B show that the compound of Formula Y and the compound of Formula Z inhibit cancer cell adhesion to vascular endothelial cells. Adhesion of human colon cancer SW620 cells to human umbilical vein endothelial cells (HUVECs) was assessed in the absence or presence of Galectin-3 (2 ug/ml) and various concentrations of the compound of Formula Y (FIG. 4A) or the compound of Formula Z (FIG. 4B). Both compounds cause dose-dependent inhibition of Galectin-3-induced SE620 cell adhesion to HUVECs.

FIG. 5A and FIG. 5B show that the compound of Formula X inhibits cancer cell invasion through vascular endothelial cells. Invasion of human melanoma ACA19 (FIG. 5A) and colon cancer SW620 (FIG. 5B) cells through human umbilical vein endothelial cells (HUVECs) was assessed in transwells in the absence or presence of the compound of Formula X (20 μM) or a Galectin-3 disaccharide inhibitor lactose (Lac, 100 μM). The compound of Formula X causes significant inhibition of Galectin-3-mediated invasion of ACA19 and SW620 cells through HUVECs. ***P<0.001, *p<0.05 (ANOVA).

FIG. 6A and FIG. 6B show that the compound of Formula Y and the compound of Formula Z inhibit cancer cell invasion through vascular endothelial cells. Invasion of human colon cancer SW620 cells through human umbilical vein endothelial cells (HUVECs) was assessed in transwells in the absence or presence of Galectin-3 (1 ug/ml), a galectin-3 disaccharide inhibitor lactose (100 uM), the compound of Formula Y and the compound of Formula Z. The compound of Formula Y (FIG. 6A) and the compound of Formula Z (FIG. 6B) both cause dose-dependent inhibition of SW620 cell invasion through HUVECs, similarly as the Galectin-3 inhibitor lactose.

FIG. 7A and FIG. 7B that the compound of Formula X inhibits human vascular cell tubule formation (angiogenesis). Vascular cell tubule formation of human umbilical vein endothelial cells (HUVECs) was assessed in the absence or presence of Galectin-3 (2 μg/ml) and the compound of Formula X (10 μM). The presence of the compound of Formula X causes significant inhibition of Galectin-3 mediated vascular cell tubule formation when vascular tubule length (FIG. 7A) and tubule numbers (FIG. 7B) were measured. ***P<0.001 (ANOVA).

FIG. 8A, FIG. 8B, FIG. 8C and FIG. 8D show that the compound of Formula Y and the compound of Formula Z inhibit vascular cell tubule formation (angiogenesis). Vascular cell tubule formation of human umbilical vein endothelial cells (HUVECs) was assessed in the absence or presence of Galectin-3 (2 μg/ml), various concentrations of the compound of Formula Y (FIG. 8A and FIG. 8C) or the compound of Formula Z (FIG. 8B and FIG. 8D). The presence of the compound of Formula Y or the compound of Formula Z causes dose-dependent inhibition of vascular cell tubule formation when vascular tubule length (FIG. 8A and FIG. 8B) and tubule numbers (FIG. 8C and FIG. 8D) were measured. *P<0.05, *** P<0.001 (ANOVA).

FIG. 9A, FIG. 9B, FIG. 9C and FIG. 9D show that the compounds of Formula X, Formula Y and Formula Z exhibit little or low toxicity. Cytotoxicity of the compound of Formula X to human colon cancer SW620 (FIG. 9A) and vascular HUVECs (FIG. 9B) cells was assessed in the absence or presence of various concentrations of the compound of Formula X by Lactate dehydrogenase (LDH) release assay. The compound of Formula X shows no detectable cytotoxicity to both human HUVEC cells and SW620 cells at 100 μM. Cytotoxicity of the compounds of Formula Y and Formula Z to human colon cancer SW620 (FIG. 9C) and vascular HUVECs (FIG. 9D) cells was assessed in the absence or presence of various concentrations of the compounds of Formula Y and Formula Z by Lactate dehydrogenase (LDH) release assay. The compounds of Formula X and Formula Z show low cytotoxicity to both human HUVEC cells and SW620 cells.

FIG. 10A, FIG. 10B and FIG. 10C show that the compound of Formula X does not affect binding of galectins-2, -4 and -8 to asialofetuin. Binding of Galectins-2 (Gal-2, FIG. 10A), -4 (Gal-4, FIG. 10B) and -8 (Gal-8, FIG. 10C) (5 μg/ml) to asialofetuin (ASF, 20 μg/ml) in the absence or presence of different concentrations of the compound of Formula X or positive control galectin inhibitor lactose was assessed by ELISA. The compound of Formula X does not affect binding of these galectin members to ASF. The disaccharide lactose dose-dependently inhibits Galectin-8 binding to ASF (positive control).

FIG. 11A, FIG. 11B and FIG. 11C. show that the compounds of Formula X, Formula Y and Formula Z inhibit galectin-3-induced secretion of pro-inflammatory cytokine TNFα from macrophages. Secretion of TNFα from THP-1 differentiated macrophages was analysed in the absence or presence of Galectin-3 (10 μg/ml) and various concentrations of Formula X (FIG. 11A), Formula Y (FIG. 11B) or Formula Z (FIG. 11C) by ELISA. Galectin-3 markedly increases secretion of TNFα and the presence of the compounds of Formula X, Formula Y and Formula Z causes dose-dependent inhibition of galectin-3-induced TNFα secretion.

FIG. 12A, FIG. 12B and FIG. 12C. show that the compounds of Formula X, Formula Y and Formula Z inhibit galectin-3-induced secretion of pro-inflammatory cytokine IL-1β from macrophages. Secretion of IL-1β from THP-1 differentiated macrophages was analysed in the absence or presence of Galectin-3 (10 μg/ml) and various concentrations of Formula X (FIG. 12A), Formula Y (FIG. 12B) or Formula Z (FIG. 12C) by ELISA. Galectin-3 markedly increases secretion of IL-1β and the presence of the compounds of Formula X, Formula Y and Formula Z causes dose-dependent inhibition of galectin-3-induced IL-13 secretion.

FIG. 13 shows inhibition of human lung cancer cell adhesion to basement matrix by the compound of Formula X (FIG. 13A), the compound of Formula Y (FIG. 13B), the compound of Formula Z (FIG. 13C), each causes dose-dependent inhibition of human lung cancer H1975 cell adhesion to matrix. **p<0.01, ***p<0.001.

FIG. 14 shows Galectin-3 Knockout of human triple-negative breast cancer MDA-MB-231 (A) and pancreatic cancer PANC1 (B) cells by CRISPR/Cas9. The galectin-3 expression in the control and knockout cells were determined by galectin-3 immunoblotting. The same blots were probed by anti-actin antibody for verification of protein loading.

FIG. 15 shows Inhibition of galectin-3-mediated human triple-negative breast cancer cell adhesion to basement matrix by the compound of Formula Y and the compound of Formula Z. The compound of Formula Y (FIG. 15A) and the compound of Formula Z (FIG. 15B) each causes dose-dependent inhibition of triple-negative breast cancer MDA-MB-231 cell adhesion to matrix. In contrast to significant inhibition on adhesion of control MDA-MB-231 cells (Con), the compound of Formula Y (FIG. 15C) and the compound of Formula Z (FIG. 15D) all lost their inhibitory effect on adhesion of the cells when galectin-3 expression in the cells was knockout by CRISPR/Cas9 (Gal3KO). ***p<0.05, p<0.01, ***p<0.001.

FIG. 16 shows Inhibition of galectin-3-mediated adhesion of human pancreatic cancer cells to basement matrix by the compounds of Formula X, Y and Z. The compound of Formula X (FIG. 16A), the compound of Formula Y (FIG. 16B), the compound of Formula Z (FIG. 16C) each causes dose-dependent inhibition of human pancreatic cancer PANC1 cell adhesion to matrix. In contrast to significant inhibition on adhesion of PANC1 cells, the compound of Formula X (FIG. 16D), the compound of Formula Y (FIG. 16E), the compound of Formula Z (FIG. 16F) all lost their inhibitory effect on cell adhesion when galectin-3 expression in the cells was knockout by CRISPR/Cas9 (Gal3KO). *p<0.05, **p<0.01, ***p<0.001.

### DETAILED DESCRIPTION

Embodiments will now be described in detail with reference to the accompanying drawings. In the following detailed description numerous specific details are set forth by way of examples in order to provide a thorough understanding of the relevant teachings. However, it will be apparent to one of ordinary skill in the art that the present teachings may be practiced without these specific details.

Throughout, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The compounds described herein may be named according to either the IIIPAC (International Union for Pure and Applied Chemistry) or CAS (Chemical Abstracts Service) nomenclature systems. It should be understood that unless expressly stated to the contrary, the terms “compounds of Formula X, Y, and/or Z” and the more general term “compounds” refer to and include any and all compounds described by and/or with reference to Formula X, Y or Z. The same should apply to any other Compounds referred to herein. It should also be understood that these terms encompasses all stereoisomers, i.e. cis and trans isomers, as well as optical isomers, i.e. R and S enantiomers, of such compounds and all salts thereof, in substantially pure form and/or any mixtures of the foregoing in any ratio. This understanding extends to pharmaceutical compositions and methods of treatment that employ or comprise one or more compounds of the Formula X and/or Formula Y and/or Formula Z, either by themselves or in combination with additional agents.

As used herein, the terms “treatment”, “treating”, “treat” or similar, refer to obtaining a desired pharmacologic and/or physiologic effect, possibly in prophylaxis as well as the alleviation of established symptoms of, or the prevention of, a disease, state, disorder or condition. The effect can be prophylactic in terms of completely or partially preventing a disease, state, disorder or condition or symptom thereof and/or can be therapeutic in terms of a partial or complete cure for a disease, state, disorder or condition and/or adverse effect attributable thereof. “Treatment”, “treating”, “treat” or similar, as used herein, cover any prevention or treatment of a disease, state, disorder or condition in a mammal, particularly in a human, and includes: (a) preventing or delaying the onset of the disease, state, disorder or condition or clinical symptoms of the disease, state, disorder or condition from occurring in a subject which can be predisposed to the disease, state, disorder or condition but has not yet been diagnosed as having it; (b) preventing or delaying the onset of the disease, state, disorder or condition or clinical symptoms of the state, disorder or condition from occurring in a subject which currently has the disease, state, disorder or condition i.e., arresting, reducing or delaying the development of the disease, state, disorder or condition or a relapse thereof (in case of maintenance treatment); (c) relieving or attenuating the disease, state, disorder or condition, i.e., causing regression of the disease, state, disorder or condition. As used herein, the term “disease” may also refer to a state, infliction, disorder or condition.

The term “therapeutic treatment” or the like refers to a treatment where the subject is known to have the disease, state, disorder or condition, e.g. there are established symptoms of a condition, prior to administration. The term “prophylactic treatment” or the like refers to a treatment where the subject is not known to have or by inflicted by the disease, state, disorder or condition prior to administration of said treatment. In some embodiments, the subject has an increased likelihood of becoming inflicted or is suspected of being inflicted prior to treatment. In some embodiments, the subject is suspected of having an increased likelihood of becoming inflicted. Said subject may be suspected of having a disease, disorder, state or condition associated with expression of Galectin-3.

The term “subject” used herein includes any human or nonhuman animal. The terms “patient” and “subject” and “subject in need treatment” may be used interchangeably throughout. The term “nonhuman animal” includes all mammals, such as nonhuman primates, sheep, rabbits, dogs, cats, cows, horses, camels, apes, monkeys, pigs.

A “therapeutically effective amount” refers to the amount of an active ingredient (such as the compound of Formula X and/or the compound of Formula Y and/or the compound of Formula Z, or any of the other compounds described herein (i.e. the compound of Formula W)), compound or composition that, when administered to a subject for treating a disease, state, disorder or condition, is sufficient to effect, enable or begin treatment steps for the disease, state, disorder or condition. The “therapeutically effective amount” will vary depending on active ingredient(s) used, the disease, state, disorder or condition and its severity and the age, weight, etc., of the subject to be treated. It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts. Throughout, “an effective amount” may be enough to inhibit Galectin-3 in vitro or in vivo. “An effective amount” may be enough to inhibit Galectin-3 so as to treating and/or preventing a disease, disorder, state or condition in a subject. “An effective amount” may mean a “therapeutically effective amount”.

### Compounds

The present invention relates to the use of four particular compounds identified as having the beneficial properties and uses as described herein. These are:

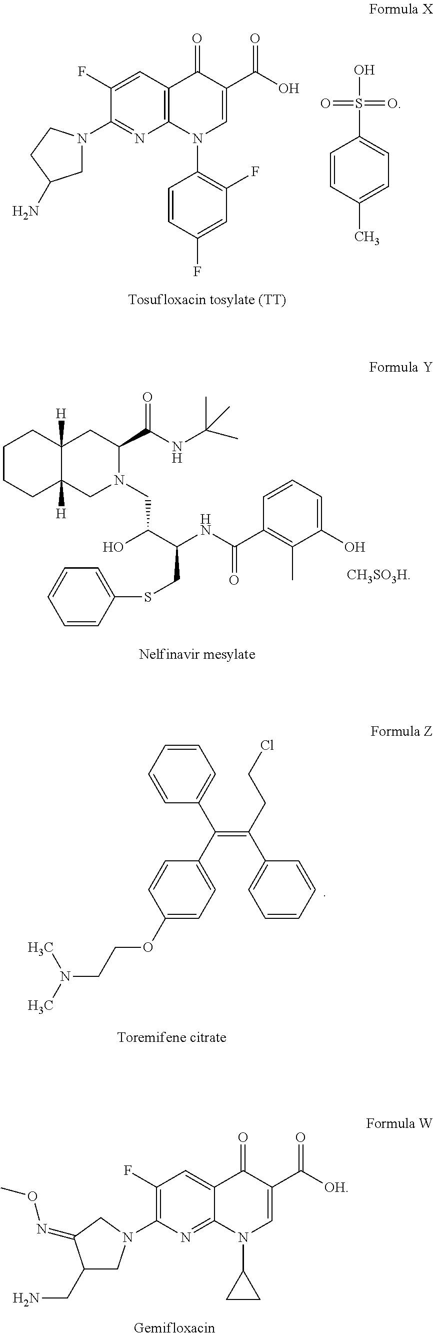

The compound of Formula X may be referred to as ‘TT’ herein. The compound of Formula Y may be referred to as ‘NM’ herein. The compound of Formula Z may be referred to as ‘TC’ herein.

The phrase “compound of the invention”, “composition of the invention”, “composition”, “compounds described herein” and “compound” means those compounds and/or compositions which are disclosed herein, both generically and specifically, specifically the compounds of Formula W, Formula X, Formula Y and/or Formula Z. Any compound or composition use described herein may refer to one or more of the compounds, or a pharmaceutically acceptable salt thereof, or the metabolized and/or hydrolysed and/or oxidised form thereof, or a composition of one or more of these compounds, in (i) use of in anything described herein; (ii) use in the manufacture of a medicament for the treatment and/or prevention of that disease, disorder, state or condition; (iii) a method of treating a disease, state, disorder or condition in a subject, the method comprising administration of a therapeutically effective amount of the compound, or a pharmaceutically acceptable salt thereof, or composition, to the subject; (iii) a method of inhibiting the target of the invention (e.g. Galectin-3), the method comprising administration of a therapeutically effective amount of one or more of the compounds, or a pharmaceutically acceptable salt thereof, or the metabolized and/or hydrolysed and/or oxidised form thereof, or a composition comprising one or more of these, to the subject or administration to a biological sample or target outside the body of a subject; (v) use of these in a method of treating a disease, state, disorder or condition in a subject, as described herein.

### Use, Medical Uses and Methods of Treatment

### Cancer and Cancer Metastasis

The compound of Formula X, or its metabolized and/or hydrolysed and/or oxidised forms, or a pharmaceutically acceptable thereof, and/or the compound of Formula Y, or its metabolized and/or hydrolysed and/or oxidised forms, or a pharmaceutically acceptable salt thereof, and/or the compound of Formula Z, or its metabolized and/or hydrolysed and/or oxidised forms, or a pharmaceutically acceptable salt thereof, or one of the other compounds described herein (i.e. the compound of Formula W), or a composition comprising one or more of the compounds of Formula X, Formula Y, of Formula Z, or one of the other compounds described herein (i.e. the compound of Formula W), or their metabolized and/or hydrolysed and/or oxidised forms, or pharmaceutically acceptable salts thereof, may be for use or used in the treatment and/or prevention of cancer. Methods, compositions and compounds are for preventing or inhibiting cancer, cancer metastasis, cancer cell proliferation, cancer reappearance after initial therapeutic treatment or intervention, tumour growth, tumour/cancer angiogenesis or immunosuppression.

Cancer is a group of diseases that may cause many different symptoms and may have many different signs, all of which may depend on the location, size and type of cancer. “Cancer” as used herein may refer to precancerous tumours or precancerous cells or conditions, tumours, cancer tumours, solid tumours, pre-metastatic cancer, tumours which have begun lead to metastatic growth, or any other such meaning known in the art. Accordingly “treatment of cancer” includes reference to or treatment of tumours of any type, reduction in tumour cell/cancer cell angiogenesis, a reduction in size of a tumour (may also be referred to as “tumour regression”), reduction in tumour volume, inhibition of immune suppression, improved clinical outcome for those treated as described herein, decrease in number of tumours, decrease in number of metastatic lesions in other tissues or organs distant from the primary tumour site, an increase in average survival time of a population of treated subjects in comparison to a population not receiving the same treatment, an increase in average survival time of a population of treated subjects in comparison to a population of untreated subjects, decrease in the mortality rate of a population of treated subjects in comparison to a population in comparison to a population of untreated subjects, a decrease in tumour growth, a decrease in tumour regrow, reduction in the rate of cellular proliferation, reduction in the proportion of proliferating cells, a decrease in size of an area or zone of cellular proliferation (e.g. the size of an area or zone of cellular proliferation, a decrease in the number or proportion of cells having an abnormal appearance or morphology), inhibition of the motility of cancer or tumour cells, containment and/or treatment of solid tumours, prevention or inhibition of tumour metastasis, reversal of tumour of tumour metastasis, an increase in tumour/cancer cell death, all of the below definitions for treatment of cancer metastasis, or any other such cancer treatment or means to measure such known in the art.

Galectin-3 activity has been associated with tumour metastasis, as described herein. Accordingly, the compounds and compositions described herein are also potentially useful agents for the prevention and/or treatment of tumour metastasis.

“Cancer metastasis” as used herein may refer to the spread of a cancer from one site in the subject body to another, secondary or different, site in the same subject's body. Accordingly “treatment of cancer metastasis” includes inhibition of the motility of cancer or tumour cells, inhibition of the dissemination and invasiveness of cancer cells leading to inhibition of metastatic tumour growth, inhibitor of the dissemination and invasiveness of cancer cells leading to improved clinical outcome for those treated as described herein, all of the above definitions for treatment of cancer”, or any other such cancer metastasis treatment or means to measure such known in the art.

For example, the incidence of metastasis can be assessed by examining relative dissemination (e.g., number of organ systems involved) and relative tumour burden in these sites. Metastatic growth can be ascertained by microscopic or macroscopic analysis, as appropriate. Tumour metastasis can be reduced by about 10%, 20%, 30%, 40%, 50%, 60%, 70%, 80%, 90%, 95% or greater.

Herein, “treatment of cancer metastasis” may be by inhibiting the activity of circulating Galectin-3 in the bloodstream, or by inhibiting the activity of Galectin-3 in one or more tumour sites. This may also be use of a Galectin-3 inhibitor such as that of the compounds of Formula X, Formula Y, Formula Z, or any of the other compounds described herein (i.e. the compound of Formula W), or a composition thereof, preventatively before potential metastasis. Prevention of cancer metastasis may comprise preventative treatment of cancer metastasis at a time of elevated risk of cancer metastasis. This may be at a time where anything may interfere with a tumour, for example surgery, use of cytotoxic drugs, radiation therapy. Treatment as described herein may reduce the risk of cancer metastasis by inhibiting Galectin-3 in the original tumour or in circulating cells comprising Galectin-3.

As used herein “outcome” or “clinical outcome” or “improved outcome” refers to the resulting course of disease and/or disease progression and can be characterized for example by recurrence, period of time until recurrence, metastasis, period of time until metastasis, number of metastases, number of sites of metastasis and/or death due to disease. For example, a positive clinical outcome may include cure, prevention of or slowing of any cancer recurrence, prevention of or slowing of metastasis and/or survival within a fixed period of time (without recurrence). A negative clinical outcome may include increased disease progression, cancer metastasis and/or death within a fixed period of time.

Cancers suitable for treatment with the compounds and compositions described herein include tumours of epithelial origin as well as blood-borne cancers. Cancers may include breast cancer [Iurisci et al, Clin Cancer Res, 2000, 6, 1389-93], particularly triple negative breast cancer [Zhang et al, PLOS one, 2014, 9, e103482], colon/colorectal/rectal cancer [colon and rectal cancers are often together called colorectal cancers due to similarities, these terms are used interchangeable herein to encompass all three terms Watanabe et al., Oncology Reports, 2011, 25, 1217-1226; Barrow et al, Clin Cancer Res, 2011 Sep. 20], head and neck cancer [Saussez et ah, Oral Oncology, 2008, 44, 86-93]; pancreatic cancer [Senapati et al., Clin Cancer Res, 2011, 17, 267-274]; lung cancer [Iurisci et al, Clin Cancer Res, 2000, 6, 1389-93]; melanoma [Vereecken et al., Melanoma Research, 2009, 19, 316-320; Vereecken et ah, Clinical and Experimental Dermatology, 2005, 31, 105-109]; thyroid cancer [Saussez et al., Thyroid, 2008, 18(7), 705-712 and Isic et ah, J Cancer Res Clin Oncol, 2010, 136, 1805-1812], Bladder cancer [Sakaki et al, J Med Invest, 2008, 55, 127-132] Gastric cancer (Kang, et al, Mol Cancer Res. 2020, 18:403-413), Ovarian cancer (Mohammed, et al, World J Surg Oncol, 2022, 20, 276), Leukaemia [Mansorunov et al, Int J Mol Sci. 2022, 23, 13846), Lymphoma (Shi et al, Front Oncol, 2022 May 23; 12:889034), Musculoskeletal tumour (Nakajima, et al, Cancer Metastasis Rev. 2021, 40:297-302), Liver cancer (Setayesh et al, Liver Res. 2020; 4:173-179) [All references are herein incorporated by reference].

Triple negative breast cancer, also known as basal-like breast cancer is a type of breast cancer where the cells lack or show low levels of estrogen receptor (ER), progesterone receptor (PR) and epidermal growth factor receptor 2 (HER2).

Galectin-3 activity has been associated with tumour angiogenesis [Califice et al., Oncogene 2004; 23, 7527-7536, herein incorporated by reference]. Accordingly, the compounds and compositions described herein are also potentially useful agents for the prevention and/or treatment of tumour angiogenesis. “Tumour angiogenesis” or just “angiogenesis” as used herein may refer to the proliferation of a network of blood vessels which supplies a tumour with a supportive microenvironment rich with oxygen and nutrients to sustain optimal growth.

Thus, also provided herein are compounds and compositions for use in, or for methods of, the inhibition, prevention and/or treatment of tumour angiogenesis. This is as described herein for treatment/prevention of cancer and cancer metastasis.

Galectin-3 is involved in tumour immune suppression by several mechanisms such as preventing T cell infiltration, apoptosis and direct interaction with tumour cells, acting as a ligand of immune checkpoint molecules and cytokines [Stillman et al, 2006, J Immunol, 176, 778-789, herein incorporated by reference], Accordingly, the compounds and compositions described herein are also potentially useful agents for reduction or inhibition of tumour immune suppression, also referred to herein as reduction or inhibition of immunosuppression or reduction or inhibition of cancer immunosuppression.

Thus there is provided the compound of Formula X, or a pharmaceutically acceptable salt thereof, and/or the compound of Formula Y, or a pharmaceutically acceptable salt thereof, and/or the compound of Formula Z, or a pharmaceutically acceptable salt thereof, or a composition of one of these compounds, or one or more of the other compounds or compositions described herein, for use in inhibiting Galectin-3, preferably for use in the treatment and/or prevention of a disease, condition, state or disorder where inhibiting Galectin-3 would be beneficial for the treatment and/or prevention of the disease condition, state or disorder, where the disease, condition, state or disorder is cancer, cancer metastasis, cancer reappearance after initial therapeutic treatment or intervention, cancer cell proliferation, tumour growth, (tumour/cancer) angiogenesis or immunosuppression. The compound may be for treating cancer, cancer metastasis, cancer reappearance after initial therapeutic treatment or intervention, cancer cell proliferation, tumour growth, (tumour/cancer) angiogenesis or immunosuppression in a subject in need thereof, for example in a method of treating such a subject or for use in treating such a subject.

### Fibrotic Diseases

The compound of Formula X, or its metabolized and/or hydrolysed and/or oxidised forms, or a pharmaceutically acceptable thereof, and/or the compound of Formula Y, or its metabolized and/or hydrolysed and/or oxidised forms, or a pharmaceutically acceptable salt thereof, and/or the compound of Formula Z, or its metabolized and/or hydrolysed and/or oxidised forms, or a pharmaceutically acceptable salt thereof, or a composition comprising one or more of the compounds of Formula X, Formula Y, of Formula Z, or their metabolized and/or hydrolysed and/or oxidised forms, or pharmaceutically acceptable salts thereof, may be for use in or used in the treatment and/or prevention of fibrosis. This may also be referred to as a fibrotic disorder, a fibroproliferative disease, or a disorder or state of wound-healing response that is out of control [Thomas A. Wynn in J Clin Invest. 2007 Mar. 1; 117(3): 524-529. doi: 10.1172/JCI31487, ‘Galectin-3: One Molecule for an Alphabet of Diseases, from A to Z’, Int J Mol Sci. 2018 February; 19(2): 379, doi: 10.3390/ijms19020379 (both of which are herein incorporated by reference)].

As discussed in the Background, Galectin-3 has been demonstrated to also be actively involved in fibrosis (such as in the lung, liver, kidney and heart) and is critical in certain conditions of heart failure. Accordingly, the compounds and compositions as described herein may be used in the treatment of fibrosis or a fibrotic disorder. Fibrosis can be defined as an out-of-control wound-healing response. It is normal for damage tissue to repair following wounds or other causes of damage, to replace dead or damaged cells. Such damage or wounds can result from various acute or chronic stimuli, including infections, autoimmune reactions, and mechanical injury. It has also been demonstrated that MSC's act via Galectin-3 pathway in treatment of kidney fibrosis and (Mesenchymal stem cells ameliorate renal fibrosis by Galectin-3/Akt/GSK3p/Snail signalling pathway in adenine-induced nephropathy rat Stem Cell Research & Therapy volume 12, Article number: 409 (2021), incorporated by reference). Further evidence suggests Belapectin (GR-MD-02) can be used in treatment of nonalcoholic steatohepatitis (NASH) (Review of Galectin-3 inhibitors in the treatment of nonalcoholic steatohepatitis, Expert Review of Clinical Pharmacology Volume 14, 2021—Issue 4, incorporated by reference).

Reference herein to “fibrosis” may refer to a fibrotic disorder, a fibrotic condition, a fibroproliferative disease, or a disorder or state of wound-healing response that is out of control. Fibrosis may refer to overgrowth, hardening, and/or scarring of various tissues and is caused by greater than normal deposition of extracellular matrix components, for example collagen.

Fibrosis may result from, and fibroproliferative diseases include or can be characterised by, excessive fibrosis (an excess of fibrous connective tissue in a tissue or organ, e.g., caused by a reparative or reactive process, e.g., in response to injury (e.g., scarring, healing) or excess fibrotic tissue arising from a single cell line (e.g., fibroma). The fibrosis, fibrotic disorder, fibrotic condition or fibroproliferative disease may be scarring or wound healing abnormalities, chronic or acute inflammation, chronic graft rejection, or a fibrotic condition affecting the heart, brain, lungs, liver, kidneys, heart or vascular system, mediastinum, bone, retroperitoneum, skin, digestive or gastrointestinal tract, connective tissue, eye or muscles. A fibrotic disorder affecting the heart or vascular system may be a cardiovascular disease, pulmonary fibroses, endomyocardial fibrosis, reactive fibrosis, arterial stiffness, old myocardial infarction, atrial fibrosis, congestive heart failure, cardiomyopathy, hypertensive heart disease (HHD), hypertension (for example pulmonary hypertension) and fibrosis associated with hypertension, atherosclerosis, restenosis (e.g. coronary, carotid, and cerebral lesions), ANCA vasculitis, Behçet's disease, anti-phospholipid syndrome, and heart disease associated with cardiac ischemic events. A fibrotic disorder affecting the brain may be brain fibrosis (e.g., glial scar). A fibrotic disorder affecting the lungs may be cystic fibrosis; idiopathic pulmonary fibrosis; coal worker's progressive massive fibrosis, popcorn lung or bronchiolitis obliterans, interstitial lung disease, cryptogenic fibrosing alveolitis, chronic fibrosing interstitial pneumonia, interstitial lung disease (ILD), diffuse parenchymal lung disease (DPLD), relapsing polychondritis, emphysema and chronic obstructive pulmonary disease (COPD), or chronic asthma. A fibrotic disorder affecting the liver may be cirrhosis, and associated conditions such as chronic viral hepatitis B or C, Wilson's disease, alcoholism, non-alcoholic fatty liver disease (NAFLD), alcoholic steatohepatitis (ASH), non-alcoholic steatohepatitis (NASH), primary biliary cirrhosis (PBC), primary sclerosing cholangitis, biliary cirrhosis or autoimmune hepatitis. A fibrotic disorder affecting the kidneys may be progressive kidney disease, diabetic nephropathy, vesicoureteral reflux, tubulointerstitial renal fibrosis; glomerulonephritis or glomerular nephritis, including focal segmental glomerulosclerosis and membranous glomerulonephritis or mesangiocapillary glomerular nephritis. A fibrotic disorder affecting the mediastinum may be mediastinal fibrosis. A fibrotic disorder affecting bone, joints or musculoskeletal system may be myelofibrosis, including primary myelofibrosis, arteritis, adhesive capsulitis, giant cell arteritis, familial Mediterranean fever, post polycythemia vera or post essential thrombocythemia myelofibrosis. A fibrotic disorder affecting the retroperitoneum may be retroperitoneal fibrosis skin. A fibrotic disorder affecting the skin may be nephrogenic systemic fibrosis, bullous pemphigoid, hydradenitis suppuritiva, discoid lupus, Dupuytren's contracture, bullous pemphigoid, keloid formation and scarring, systemic sclerosis or scleroderma, sine scleroderma, or a scleroderma-like disorder. A fibrotic disorder affecting the digestive or gastrointestinal tract may be fibrotic intestinal disorder, inflammatory bowel disease, ulcertative colitis or Crohn's disease. A fibrotic disorder affecting the connective tissue may be arthrofibrosis or capsulitis. A fibrotic disorder affecting the eye may be macular degeneration, Graves' ophthalmopathy, ocular fibrosis following surgery or pseudoexfoliation syndrome glaucoma. A fibrotic disorder affecting muscles may be polymyositis. Other multi-organ or other organ affecting fibrotic disorders include scarring or wound healing abnormalities, chronic graft rejection, sarcoidosis, Peyronie's disease or oral fibrosis.

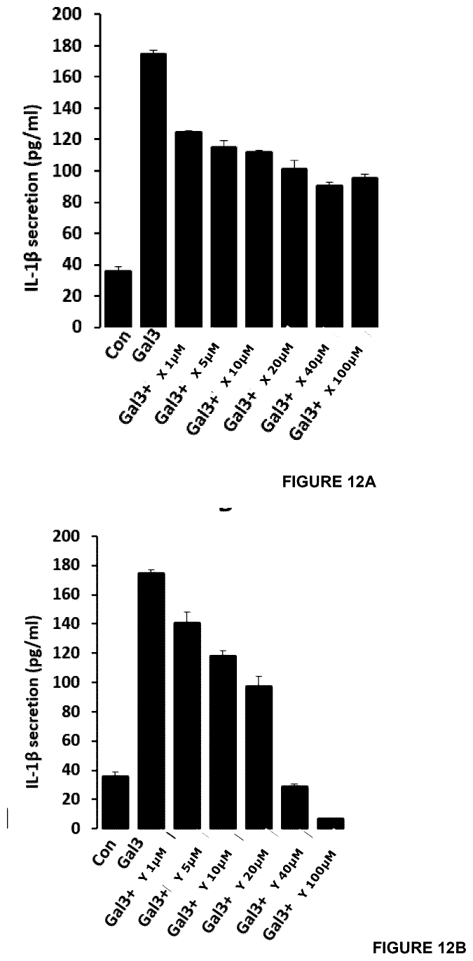

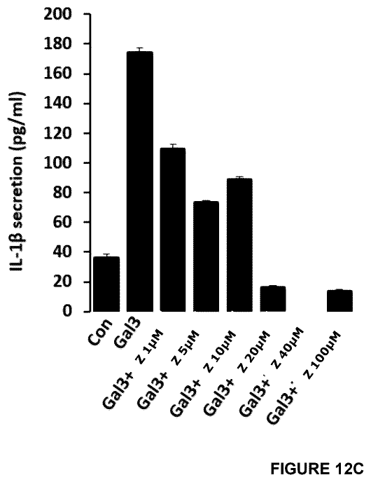

The effect of the compounds described herein on inhibition of Galectin-3-mediated secretion of pro-inflammatory cytokines from macrophages (FIG. 12) further demonstrates how these compounds may be utilised to treat fibrosis and related disorders. Secretion of pro-inflammatory cytokines is one of the fundamental mechanisms in Galectin-3-mediated pathogenesis of fibrosis, inhibition of this will inhibit or treat fibrosis.

Thus there is provided the compound of Formula X, or a pharmaceutically acceptable salt thereof, and/or the compound of Formula Y, or a pharmaceutically acceptable salt thereof, and/or the compound of Formula Z, or a pharmaceutically acceptable salt thereof, or a composition of one of these compounds, or one or more of the other compounds or compositions described herein, for use in inhibiting Galectin-3, preferably for use in the treatment and/or prevention of a disease, condition, state or disorder where inhibiting Galectin-3 would be beneficial for the treatment and/or prevention of the disease condition, state or disorder, where the disease, condition, state or disorder is fibrosis, a fibrotic disorder, a fibrotic condition, a fibroproliferative disease, or a disorder, condition or state of wound-healing response that is out of control. One or more of the compounds may be used for reducing fibrosis or a fibrosis-related condition in a subject in need thereof, for example in a method of treating such a subject or for use in treating such a subject.

### Inflammation

The compound of Formula X, or its metabolized and/or hydrolysed and/or oxidised forms, or a pharmaceutically acceptable thereof, and/or the compound of Formula Y, or its metabolized and/or hydrolysed and/or oxidised forms, or a pharmaceutically acceptable salt thereof, and/or the compound of Formula Z, or its metabolized and/or hydrolysed and/or oxidised forms, or a pharmaceutically acceptable salt thereof, or one of the other compounds described herein, or a composition comprising one or more of the compounds of Formula X, Formula Y, of Formula Z, or one of the other compounds described herein (i.e. the compound of Formula W), or their metabolized and/or hydrolysed and/or oxidised forms, or pharmaceutically acceptable salts thereof, may be for use in or may be used in the treatment and/or prevention of inflammation or one or more inflammation related disorders.

Inflammation results from the stimulation of the body by internal or external inflammatory factors, or a combination or both, and the resulting effects. This can be the basis of many chronic diseases, including infectious diseases, autoimmune diseases, or degenerative/ageing related diseases such as Alzheimer's disease.

Inflammation can also be linked to the induction, promotion, malignant transformation, invasion and metastasis of cancer. Further, particularly, inflammation is closely linked to fibrosis (Pathological fibrosis results from abnormal tissue repair, from cellular stress, chronic, inflammation and/or severe tissue damage). Chronic inflammation with resultant fibrosis, loss of tissue architecture, scarring and organ failure is a major part of the pathogenesis of many chronic diseases and is a major cause of morbidity and mortality.

Galection-3 is also known to be expressed in many cells involved in starting or mediating inflammatory response, such as macrophages, monocytes, dendritic cells, eosinophils, mast cells (activation), natural killer cells, neutrophil activation and adhesion, opsonization of apoptotic neutrophils, as well as T lymphocytes and activated B lymphocytic cells phagocytic macrophages (including being a macrophage activation marker).

Thus there is provided a compound of Formula X and/or Formula Y and/or Formula Z, and/or pharmaceutically acceptable salts thereof, for use in inhibiting Galectin-3, preferably for use in the treatment and/or prevention of a disease, condition, state or disorder where inhibiting Galectin-3 would be beneficial for the treatment and/or prevention of the disease condition, state or disorder, where the disease, condition, state or disorder is inflammation, an inflammation disease or an inflammation-related disease, condition, state or disorder. The compound may be to reducing inflammation in a subject in need thereof, including but not limited to a subject having an autoimmune disease, for example in a method of treating such a subject or for use in treating such a subject.

Inflammation and inflammation related diseases, disorders, states and conditions include, but are not limited to, caused by acute or chronic organ transplant rejection, graft-versus-host disease, inflammatory bowel disease, inflammatory skin disease, multiple sclerosis, arteriosclerosis, pancreatitis, acute bronchitis, chronic bronchitis, Alzheimer's disease, inflammatory lung disease, inflammatory skin disease, Acute bronchiolitis, folliculitis, chronic bronchiolitis, musculoskeletal pain or connective tissue inflammation, osteoarthritis, gout, spondyloarthropathy, Reiter's syndrome, psoriatic arthropathy, and inflammation caused by bacterial, fungal, and viral infections one or more diseases, inflammatory symptoms caused by autoimmune diseases, the autoimmune diseases including, but are limited to, ulcerative colitis (UC) and Crohn's disease (CD), lupus erythematosus, hyperthyroidism, IgA nephritis, type I or type II diabetes and its complications, dry eye syndrome, rheumatoid arthritis, simple obesity, ankylosing spine inflammation, bronchial asthma, neurodermatitis, ulcerative colitis, oral ulcers, psoriasis, vitiligo, Behçet's disease, autoimmune iridocyclitis, autoimmune eczema, autoimmune uveitis, One or more of autoimmune conjunctivitis, autoimmune dry eye, autoimmune glaucoma, autoimmune cataract, allergic rhinitis, irritable bowel syndrome, and pruritus. Other inflammation and inflammation related disorders include, but are not limited to, atherosclerosis, dermatitis, diverticulitis, fibromyalgia, hepatitis, irritable bowel syndrome, nephritis, Parkinson's disease, chronic inflammation, one or more neurodegenerative diseases or disorders caused by tumours caused by chronic inflammation and aging caused by chronic inflammation. The tumours caused by the chronic inflammation include but are not limited to melanoma, or tumours related to breast, liver, lung, skin, oral cavity, esophagus, stomach, intestinal tract, kidney, prostate, brain, nervous system, bladder, lymph, pancreas, etc. Tumours, especially tumours of the digestive tract, such as intestinal tumours.

Thus, there is provided a compound of Formula X and/or Formula Y and/or Formula Z, and/or pharmaceutically acceptable salts thereof, or a composition of one of these compounds, or one or more of the other compounds or compositions described herein, for use in inhibiting Galectin-3, preferably for use in the treatment and/or prevention of a disease, condition, state or disorder where inhibiting Galectin-3 would be beneficial for the treatment and/or prevention of the disease condition, state or disorder, where the disease, condition, state or disorder is inflammation or one or more inflammation related disorders. One or more of the compounds may be used to treat inflammation or one or more inflammation related disorders in a subject in need thereof, including but not limited to a subject having one or more inflammation related disorders, for example in a method of treating such a subject or for use in treating such a subject.

### Diabetes

The compound of Formula X, or its metabolized and/or hydrolysed and/or oxidised forms, or a pharmaceutically acceptable thereof, and/or the compound of Formula Y, or its metabolized and/or hydrolysed and/or oxidised forms, or a pharmaceutically acceptable salt thereof, and/or the compound of Formula Z, or its metabolized and/or hydrolysed and/or oxidised forms, or a pharmaceutically acceptable salt thereof, or one of the other compounds described herein (i.e. the compound of Formula W), or a composition comprising one or more of the compounds of Formula X, Formula Y, of Formula Z, or one of the other compounds described herein (i.e. the compound of Formula W), or their metabolized and/or hydrolysed and/or oxidised forms, or pharmaceutically acceptable salts thereof, may be for use in or may be used in the treatment and/or prevention of diabetes.

Diabetes mellitus is the scientific name for diabetes, but it is more commonly known simply as diabetes. Diabetes is classified as a chronic inflammatory disease, and Gal-3 is thought to have a proactive role in diabetes, particular in type 2 diabetes mellitus (T2DM). Gal-3 is thought to be involved in the regulation of glucose homeostasis, acting in adipose tissue and pancreatic islets, and thus possibly participating in the pathogenesis of obesity and T2DM. [Galectin-3: One Molecule for an Alphabet of Diseases, from A to Z′, Int J Mol Sci. 2018 February; 19(2): 379, doi: 10.3390/ijms19020379 (herein incorporated by reference).]

Insulin resistance is a hallmark of Type 2 diabetes and plays a central role in this metabolic disease. Galectin-3 has been shown to cause cellular and systemic insulin resistance. This effect of Galectin-3 is believed partly due to Galectin-3 binding to the insulin receptor (IR) and inhibit IR downstream signalling (Li et al, Hematopoietic-Derived Galectin-3 Causes Cellular and Systemic Insulin Resistance. Cell. 2016; 167:973-984).

Thus there is provided a compound of Formula Z and/or Formula Y and/or Formula Z, and/or pharmaceutically acceptable salts thereof, or a composition of one of these compounds, or one or more of the other compounds or compositions described herein, for use in inhibiting Galectin-3, preferably for use in the treatment and/or prevention of a disease, condition, state or disorder where inhibiting Galectin-3 would be beneficial for the treatment and/or prevention of the disease condition, state or disorder, where the disease, condition, state or disorder is diabetes. The compound may be used to treat diabetes in a subject in need thereof, including but not limited to a subject having diabetes, for example in a method of treating such a subject or for use in treating such a subject. The diabetes may be type I or type II diabetes and/or related diabetic-associated diseases or conditions, such as obesity.

Other Disease, State, Disorder or Conditions which are Affected and/or Mediated by Galectin-3

Galectin-3 is known to play a role in several other diseases, states, disorders, conditions or related pathogenesis. A comprehensive outline of this can be found in ‘Galectin-3: One Molecule for an Alphabet of Diseases, from A to Z’, Int J Mol Sci. 2018 February; 19(2): 379, doi: 10.3390/ijms19020379 (herein incorporated by reference).

Further conditions mediated by Galectin-3 include Asthma (Gal-3 has a role in the pathophysiological mechanisms in immune response and, in particular, in asthma, Gal-3 in asthma is related to its function in the recruitment, activation and removal of neutrophils), Atherosclerosis (Gal-3 can be detected close to a lipid core or near areas with fibrosis, neovascularization, calcification, hemorrhage, or thrombosis. and Gal-3 mRNA can be detected in advanced atherosclerotic lesions from carotid and lower limb arteries), Atopic Dermatitis (AD, Gal-3 is a pro-inflammatory mediator of skin inflammation in atopic skin disease and that is required for the development of the Th2 inflammatory response to epicutaneously-introduced antigens through its effects on both DCs and T cells, and Gal-3 is thought to play a critical role in the development of the allergic inflammatory response in AD), Cerebral infarction (levels of Gal-3 have been reported to increase in ischemic brain damage, Gal-3 serum levels were found to be increased in clinically-suspected ischemic stroke patients with normal CCT, seen in the emergency department), COPD (Gal-3 has been demonstrated to be higher in patients with COPD with elevated systolic pulmonary artery pressure compared to healthy controls), Degenerative Aortic Stenosis (Gal-3 myocardial expression was increased in aortic stenosis patients with cardiac hypertrophy and relatively depressed ejection fraction compared to aortic stenosis subjects with LV hypertrophy and normal or elevated ejection fraction), Endometriosis (Endometriosis is caused by a dysregulation of inflammatory and vascular signalling and Gal-3 plays a relevant role in both these processes and it is overexpressed in endometriotic tissue, higher levels of Gal-3 can be detected in the peritoneal fluids from women with endometriosis), Encephalitis (Gal-3 is involved in inflammatory response and in the interaction with extracellular matrix, a potential role has been postulated in various types of CNS inflammation and in particular in viral encephalitis), Gastritis (Numerous infectious agents, such as Trypanosma cruzi, human immunodeficiency virus-1 and the human T-lymphotropic virus-1, have been reported to be able to induce Gal-3 upregulation.), HIV infection (Gal-3 has been found to be associated with early stages of HIV infection, in particular during transport and/or splicing of HIV mRNA), Interstitial lung disease (Gal-3 is responsible for a promoting effect on IPF, Gal-3 was increased in the serum of patients affected by IPF, and higher Gal-3 concentrations were associated with decreased lung volumes and altered gas exchange and considering the lack of effective treatment for this pulmonary condition, it has been postulated that Gal-3 may represent a possible new target for treatment), Juvenile Idiopathic Arthritis (observations indicate that Gal-3 plays a crucial role in the pathophysiology of Juvenile idiopathic arthritis), CVD mortality (Gal-3 concentrations are associated with incident HF and mortality in the general population), Non-alcoholic steatohepatitis (NASH, Gal-3 appears to be a critical regulator of liver fibrosis attempts have been made to inhibit its activity, link between Gal-3 and hepatic fibrosis was established when mice that lack the Gal-3 gene were found to be resistant to liver fibrosis induced by toxin administration), Obesity (experimental evidence, using Gal-3 knockout mice indicate that in vivo Gal-3 administration causes glucose intolerance and insulin resistance whereas in vitro treatment with selective small molecule Gal-3 inhibitors can directly induce decreased insulin sensitivity in myocytes, hepatocytes, and adipocytes. Such experiments suggest a possible interesting link between inflammation and decreased insulin sensitivity), Pneumonia (Gal-3 is involved in the recruitment of neutrophils during lung infections with Streptococcus pneumoniae), Pulmonary hypertension (Gal-3 is able to modulate fibroblast and endothelial behaviour acting on fibroblast and vascular endothelial growth factors, Gal-3 seems to play a key role in acute and chronic inflammatory responses to endothelial injury, vascular remodeling and fibrosis in PAH), Plaque Psoriasis (Gal-3 is associated with keratinocyte differentiation andmaturation, plaque psoriasis is one of the most prevalent autoimmune skin diseases characterized by the presence of well-demarcated red silvery scales on the skin surface and Gal-3 was expressed in a high proportion of Langerhans cells from skin punch biopsies, obtained from lesional plaque-type psoriatic skin), Q Fever (The etiologic agent of Q-fever is a highly infectious human pathogen named Coxiella burnetiid, Gal-3 may function as a danger receptor for membrane damages and the role of the expression of Galectins, and in particular of Gal-3 in the lysosomes of the infected cells appears to be crucial, with a possible role of Gal-3 in the mechanisms of autophagy during membrane repair), Rheumatoid Arthritis (RA, Gal-3 promotes inflammation in RA and it has been shown in animal models and human studies that Gal-3 plays a crucial role in the development of arthritis), Systemic Sclerosis (elevated serum Gal-3 levels in patients show a clear association with the signs of the advanced organ sclerosis and with the laboratory parameters of inflammation), Urinary tract infections and renal infections (Gal-3 was found to play a crucial role in bacterial infections, and in particular in infections due to Proteus mirabilis, a bacterium frequently detected in the urinary tract, Gal-3 influences adhesion of such bacterium to the extracellular surface of the plasma membrane of different kidney epithelial cell lines and its crucial role in Proteus mirabilis infection was demonstrated using specific monoclonal antibodies directed against Gal-3, showing Gal-3 plays a key role of in renal pathology due to infections), Covid-19 (Many studies have now demonstrated the involvement of Galectin-3 in Covid-19 infection and progression, e.g. Immunopathology of Galectin-3: an increasingly promising target in COVID-19 (F1000Research 2020, 9:1078 Last updated: 6 Feb. 2023), clinical trials have also been run with galectin-3 inhibitors treating COVID16, see e.g. Sigamani et al, An Oral Galectin Inhibitor in COVID-19-A Phase Randomized Controlled Trial. Vaccines (Basel) and Computational Study of Potential Galectin-3 Inhibitors in the Treatment of COVID-19 (Biomedicines 2021, 9, 1208)), sepsis is also mediated and/or exacerbated by Galectin-3 (see e.g. ‘Serum galectin-3 levels predict poor prognosis in sepsis and septic shock patients’, Rev Assoc Med Bras (1992). 2023; 69(8): e20220940; and ‘Galectin-3 aggravates experimental polymicrobial sepsis by impairing neutrophil recruitment to the infectious focus’, Journal of Infection, Volume 77, Issue 5, November 2018, Pages 391-397), Thrombosis and Venous Thrombosis (Gal-3 is associated with thrombogenesis and in particular, it is characterized by prothrombotic and pro-inflammatory properties in the context of experimental venous thrombosis, serum levels of Gal-3 and of Gal-3 binding protein are elevated in the course of venous thrombosis in both mice and humans, studies have also shown the use of Galectin-3 inhibitors as novel antithrombotic drugs—European Heart Journal, Volume 43, Issue 37, 1 Oct. 2022, Pages 3575-3577, https://doi.org/10.1093/eurhearti/ehac128; and Galectin 3 has been shown to enhance platelet aggregation and thrombosis via Dectin-1 activation—European Heart Journal, Volume 43, Issue 37, 1 Oct. 2022, Pages 3556-3574, https://doi.org/10.1093/eurheartj/ehac034), Wound Healing (Gal-3 is able to modulate cell migration through its effects on cell adhesion and its interactions with cell-matrix and is considered a key player in the re-epithelialization of wounds in many different tissues), Cardiac syndrome X (CSX, Serum levels of Gal-3 were measured in patients affected by CSX and were significantly higher in the CSX patients in comparison with the healthy controls), Yeast infection-Candidiasis (inhibition of Gal-3 in neutrophils may be a promising therapeutic strategy for systemic Candida infection, the inhibition of Gal-3 is able to inhibit neutrophil reactive oxygen species production and enhance the ability of neutrophils to kill when encountering “hard-to-kill” Candida) and Zoster-related pain (allodynia, Gal-3 is involved in the process of Wallerian degeneration distal to the injury and subsequent axonal regrowth and functional re-innervation and Gal-3 has emerged as a new target for the treatment and clinical management of peripheral nerve injury-induced neuropathic pain). All are explained in more detailed in the above referenced literature. These diseases, states, disorders or conditions may be referred to as disease, state, disorder or conditions affected and/or mediated by Galectin-3.

Thus, there is provided the compound of Formula X, or its metabolized and/or hydrolysed and/or oxidised forms, or a pharmaceutically acceptable thereof, and/or the compound of Formula Y, or its metabolized and/or hydrolysed and/or oxidised forms, or a pharmaceutically acceptable salt thereof, and/or the compound of Formula Z, or its metabolized and/or hydrolysed and/or oxidised forms, or a pharmaceutically acceptable salt thereof, or one of the other compounds described herein (i.e. the compound of Formula W), or a composition comprising one or more of the compounds of Formula X, Formula Y, of Formula Z, or one of the other compounds described herein (i.e. the compound of Formula W), or their metabolized and/or hydrolysed and/or oxidised forms, or pharmaceutically acceptable salts thereof, may be for use in or may be used in inhibiting Galectin-3, preferably for use in the treatment and/or prevention of a disease, condition, state or disorder where inhibiting Galectin-3 would be beneficial for the treatment and/or prevention of the disease condition, state or disorder, where the disease, condition, state or disorder is one of these.

### Combination Therapies

The compositions and compounds defined herein may be used alone to provide a therapeutic effect. For example, for cancer treatment, the compositions and compounds defined herein may also be used in combination with one or more of surgery and/or additional anti-tumour agents, anti-metastatic agents, chemotherapy agents/chemotherapy, and/or radiotherapy agents/radiotherapy, anti-fibrosis/fibrotic agents, anti-inflammation/inflammatory agents, one or more other Galectin inhibitor and/or immunotherapeutic agents/immunotherapy.

Such chemotherapy may include one or more of the following categories of anti-tumour agents:
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-I-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbBI antibody cetuximab [Erbitux, C225] and any growth factor or growth factor receptor antibodies (Stern et al. Critical reviews in oncology/haematology, 2005, Vol. 54, ppl 1-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (RI 15777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense; and/or
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Such immunotherapy may include one or more of the following categories of immunotherapeutic agents: immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches to prevent activation of immune checkpoint molecules such as PD-1 and lymphocyte activation gene-3 (LGA3), approaches using transfected immune cells such as cytokine-transfected dendritic cells, approaches using cytokine-transfected tumour cell lines and approaches using anti-idiotypic antibodies.

For example, for fibrosis treatment, the compositions and compounds defined herein may also be used in combination with one or more of surgery and/or additional anti-fibrosis/fibrotic agents. The anti-fibrosis/fibrotic agents may be any described in Z. Manyu, Targeting fibrosis: mechanisms and clinical trials, Signal Transduction and Targeted Therapy volume 7, Article number: 206 (2022) (herein incorporated by reference). For example, TGF-β/TGFβR targeting agents, FGF targeting agents, RTKs targeting agents, CTGF targeting agents, PI3K targeting agents, JAK targeting agents, WNT/β-catenin targeting agents, ASK targeting agents, MAPK targeting agents, LOXL targeting agents, PPAR targeting agents, FXR targeting agents, TLR targeting agents, GLP/GIP targeting agents, CFTR targeting agents, HDAC targeting agents, THRP targeting agents, CCR targeting agents, MPC targeting agents, SCD targeting agents, ATX targeting agents, FATP5 targeting agents, ACC targeting agents, PDE targeting agents, AMPK targeting agents, PDE targeting agents, AMPK targeting agents, MMP targeting agents, A3AR targeting agents, FASN targeting agents, Bioidentical testosterone targeting agents, Stem cell targeting agents, HSP targeting agents, CD targeting agents, ileal bile acid transport targeting agents, aldosterone receptor targeting agents, GPR targeting agents, ROCK2 targeting agents, BAFFR targeting agents, LPA1 targeting agents, Telomerase targeting agents, KHK targeting agents, calpain targeting agents, P selectin targeting agents, SMO targeting agents, Bcl-2 targeting agents, BET family targeting agents, ENaC targeting agents, DNase I targeting agents, AA/DHA imbalance targeting agents, Neutrophil elastase targeting agents, leukotriene B4 targeting agents, CDK targeting agents, LSD targeting agents, MDM2 targeting agents, PLK1 targeting agents, IL-la targeting agents, HSD17B13 targeting agents, MOTS-c targeting agents, IFN-γ targeting agents, Autotaxin targeting agents, Glutathione dependent PGD synthase targeting agents, Arginase targeting agents, GSNOR targeting agents, Pim kinase inhibitor targeting agents and PRMT targeting agents.

For example, the TGF-β/TGFPβR targeting agent may be Pirfendione, Hydronidone, HEC-585, PLN-74809, BG00011, IDL-2965, TRK-250, Luspatercept, Sotatercept, AVID200, the FGF targeting agent may be BI089-100, Efruxifermin, Pegbelfermin, Aldafermin, the RTKs targeting agent may be Nintedanib, ZSP1603, MK-3655, the CTGF targeting agent may be, Pamrevlumab, the PI3K targeting agent may be, Parsaclisib, Omipalisib, HEC-68498, Buparlisib, Umbralisib, the JAK targeting agent may be Ruxolitinib, Fedratinib, Momelotinib, Pacritinib, Jaktinib, Itacitinib, Ilginatinib, the WNT/β-catenin targeting agent may be SM04646, PRI-724, the ASK, MAPK targeting agent may be Selonsertib, CC-90001, MG-S-2525, the LOXL targeting agent may be Epeleuton, Tipelukast, PAT-1251, PXS-5382A, the PPAR targeting agent may be Elafibranor, Saroglitazar, Lanifibranor, Pemafibrate, ZSP0678, the FXR targeting agent may be Obeticholic Acid, Cilofexor, Nidufexor, TERN-101, Vonafexor, EDP-305, Tropifexor, the TLR targeting agent may be JKB-121, JKB-122, the GLP/GIP targeting agent may be Semaglutide, Tirzepatide, Cotadutide, HM-15211, the CFTR targeting agent may be Elexacaftor, Ivacaftor, GLPG1837, FDL169, Olacaftor, VX-152, MRT5005, GLPG2737, Nesolicaftor, VX-121, ABBV-3067, ELX-02, Eluforsen, Dirocaftor, FDL176, Posenacaftor, GLPG2451, the HDAC targeting agent may be Panobinostat, Pracinostat, the THRP targeting agent may be Resmetirom, VK2809, the CCR targeting agent may be Cenicriviroc, the MPC targeting agent may be Azemiglitazone potassium, Deuterium-Stabilized (R)-Pioglitazone, the SCD targeting agent may be Aramchol, the ATX targeting agent may be Ziritaxestat, the FATP5 targeting agent may be Ursodiol, the ACC targeting agent may be PF-05221304, Firsocostat, the PDE targeting agent may be ZSP1601, Epeleuton, Tipelukast, Ensifentrine, the AMPK targeting agent may be PXL-770, the AMPK targeting agent may be ALS-L1023, the MMP targeting agent may be ALS-L1023, the A3AR targeting agent may be Namodenoson, the FASN targeting agent may be TVB-2640, the Bioidentical testosterone targeting agent may be LPCN 1144, the Stem cell targeting agent may be HepaStem, the HSP targeting agent may be BMS-986263, PU-H71, the CD targeting agent may be may be Foralumab, Tagraxofusp, the ileal bile acid transport targeting agent may be Elobixibat, the aldosterone receptor targeting agent may be Apararenone, the GPR targeting agent may be RVT1601, GLPG-1205, PBI-4050, the ROCK2 targeting agent may be Belumosudil, the BAFFR targeting agent may be Ianalumab, the LPA1 targeting agent may be BMS-986278, the Telomerase targeting agent may be Imetelstat, the KHK targeting agent may be PF-06835919, the calpain targeting agent may be BLD-2660, the P selectin targeting agent may be Crizanlizumab, the SMO targeting agent may be Sonidegib, the Bcl-2 targeting agent may be Navitoclax, the BET family targeting agent may be Pelabresib, the ENaC targeting agent may be BI-1265162, β-1037, QBW276, IONIS-ENaCRx, AZD5634, BI 443651, Idelalisib, the DNase I targeting agent may be AIR DNase, the AA/DHA imbalance targeting agent may be Fenretinide, the Neutrophil elastase targeting agent may be Lonodelestat, CHF 6333, the leukotriene B4 targeting agent may be Acebilustat, the CDK targeting agent may be Seliciclib, Ribociclib, the LSD targeting agent may be Bomedemstat bis-tosylate, the MDM2 targeting agent may be KRT-232, the PLK1 targeting agent may be Rigosertib, the IL-la targeting agent may be Bermekimab(MABp1), the HSD17B13 targeting agent may be ARO-HSD, the MOTS-c targeting agent may be CB4211, the IFN-γ targeting agent may be Interferon gamma, the Autotaxin targeting agent may be BBT-877, the Glutathione dependent PGD synthase targeting agent may be ZL-2102, the Arginase targeting agent may be CB-280, the GSNOR targeting agent may be N-6022, the Pim kinase inhibitor targeting agent may be TP-3654 or the PRMT targeting agent may be PRT-543.

For example, for inflammation treatment, the compositions and compounds defined herein may also be used in combination with one or more of surgery and/or additional anti-inflammation/inflammatory agents. For example, anti-inflammatory drugs, also called anti-inflammatories. For example, Nonsteroidal anti-inflammatory drugs (NSAIDs), Antileukotrienes and Immune selective anti-inflammatory derivatives (ImSAIDs).

NSAIDs include aspirin, ibuprofen, naproxen, diclofenac, celecoxib, mefenamic acid, etoricoxib and indomethacin. Antileukotrienes are also known as leukotriene modifier and leukotriene receptor antagonists, include leukotriene-related enzyme inhibitors (arachidonate 5-lipoxygenase) or leukotriene receptor antagonist (cysteinyl leukotriene receptors), including montelukast, zafirlukast, and pranlukast, and 5-lipoxygenase inhibitors, like zileuton and Hypericum perforatum. Immune Selective Anti-Inflammatory Derivatives (ImSAIDs) are a class of peptides that have anti-inflammatory properties, including Salivary gland derived peptides.

The compositions and compounds defined herein may also be used in combination with one or more other Galectin inhibitors. The compounds of Formula X, Formula Y, Formula Z or any of the other compounds described herein (i.e. the compound of Formula W) may be used in combination with each other. Or, one or more of the compounds of Formula X, Formula Y, Formula Z or any of the other compounds described herein (i.e. the compound of Formula W) may be used in combination with another Galectin inhibitor, for example another Galectin-3 inhibitor, for example Belapectin, Selvigaltin, GB1211, GB0139, GB1107, G3-C12 TFA, β-Lactose, G3-C12, Galectin-3-IN-1 or Galectin-3 antagonist 1. Or a Galectin-1 inhibitor for example OTX008 or Apoptosis inducer 8. Or a multiple Galectin type inhibitor such as Thiodigalactoside.

Compositions and compounds defined herein may also be applied as a sole therapy or may involve, in addition to the compounds/compositions described herein, treatment with surgery, radiotherapy, chemotherapy, immunotherapy or another treatment such as with an anti-metastatic agent.

Such treatment may be achieved by way of the simultaneous, sequential or separate dosing of the individual components of the treatment. Such combination products employ the compounds/compositions described herein within the dosage range described herein and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

The compounds or compositions described herein may be used in combination with surgical treatments. For example, surgical treatment of cancer and/or cancer metastasis and/or fibrosis and/or inflammation and/or diabetes. A surgical removal of cancer or fibrotic material may be, for example, carried out by cryosurgery.

Additionally, or alternatively, the compounds or compositions described herein may be used as adjuvant in the treatment of a Galectin-3-mediated disease, disorder, state or condition. An adjuvant is an agent provided to a patient together with, or after a “main” treatment or therapy. For example, for cancer treatment, the compounds or compositions described herein may be provided after a “main” anti-cancer therapy, such as surgical removal of cancer, in order to prevent the return of cancer after the main therapy. This may be after or as part of a “main” treatment, for example surgical removal of a tumour or tumour debulking. This could also be for example surgical removal of fibrotic tissue.

### Compositions

The compositions according to the aspects of the invention may further comprise one or more pharmaceutically or cosmetically acceptable ingredients or excipients. Pharmaceutically acceptable ingredients are well known to those skilled in the art, and include, but are not limited to, pharmaceutically acceptable carriers, adjuvants, excipients, diluents, fillers, buffers, preservatives, anti-oxidants, lubricants, stabilisers, solubilisers, surfactants (e.g. wetting agents), masking agents, colouring agents, fragrance agents and penetration agents. Conventional procedures for the selection and preparation of suitable pharmaceutical Formulations are described in, for example, “Pharmaceuticals—The Science of Dosage Form Designs”, M. E. Aulton, Churchill Livingstone, 1988.

In some embodiments, the compositions may be provided as a suspension in a pharmaceutically or cosmetically acceptable excipient, diluent or carrier.

The compositions described herein may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions described herein may be obtained by conventional procedures using conventional pharmaceutical excipients, well known in the art. Thus, compositions intended for oral use may contain, for example, one or more colouring, sweetening, flavouring and/or preservative agents.

The in vivo effects of a compound or composition described herein may be exerted in part by one or more metabolites that are formed within the human or animal body after administration of a compound or composition of the invention.

Further information on the preparation of the compounds described herein is provided in the Examples section. The general reaction schemes and specific methods described in the Examples form a further aspect of the invention.

The compounds described herein can be isolated and purified using techniques well known in the art.

In general, salts can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

The compounds described herein may exist in a single crystal form or in a mixture of crystal forms or they may be amorphous. Thus, the compounds described herein intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

The processes defined herein may further comprise the step of subjecting the compounds described herein to a salt exchange, particularly in situations where one or more of the compounds described herein are formed as a mixture of different salt forms. The salt exchange suitably comprises immobilising one or more of the compounds described herein on a suitable solid support or resin, and eluting the compounds with an appropriate acid to yield a single salt of one or more of the compounds described herein n.

In a further aspect of the invention, there is provided a compound described herein obtainable by any one of the processes defined herein.

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O, ¹⁸F and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

It is also to be understood that certain compounds described herein may exhibit polymorphism, and that the invention encompasses all such forms that possess the desirable properties describes herein.

Compounds described herein may exist in a number of different tautomeric forms and references to compounds described herein include all such forms. For the avoidance of doubt, where a compound can exist in one of several tautomeric forms, and only one is specifically described or shown, all others are nevertheless embraced by the compounds described herein. Examples of tautomeric forms include keto-, enol-, and enolate-forms, as in, for example, the following tautomeric pairs: keto/enol, imine/enamine, amide/imino alcohol, amidine/amidine, nitroso/oxime, thioketone/enethiol, and nitro/aci-nitro.

The compounds described herein may be and used per se in the methods disclosed herein or may be used in the form of a pharmaceutically acceptable salt. It is also to be understood that certain compounds described herein may exist in solvated as well as unsolvated forms such as, for example, hydrated forms. It is to be understood that the invention encompasses all such solvated forms that possess the desirable properties describes herein.

### Routes of Administration and Dosage

The compounds and compositions described herein may be administered in a number of ways depending on the desired site of action. The compounds and compositions, according to the methods described herein, are administered using any amount and any route of administration effective for treating or lessening the severity of a disorder provided above. The exact amount required will vary from subject to subject, depending on the species, age, and general condition of the subject, the severity of the disease, disorder, condition or state, the particular agent, its mode of administration, and the like.

Compounds and/or pharmaceutically acceptable salts thereof, or solvates thereof, or hydrates thereof, or compositions described herein may be administered orally, parenterally, by inhalation spray, topically, rectally, nasally, buccally, vaginally or via an implanted reservoir. Preferably, they are administered orally, intraperitoneally or intravenously. Most preferably, pharmaceutically acceptable compositions described herein are formulated for oral administration. Exemplary oral dosage forms are capsules, tablets, aqueous suspensions or solutions. Such compositions are prepared according to techniques well-known in the art of pharmaceutical Formulation.

Suitably, the compounds and compositions described herein may be administered by injection. Injections may be, for example, intravenous, intra-arterial, intradermal, subcutaneous, intraperitoneal, intracerebral, intracerebroventricular or intrathecal. Such injections may be continuous over a period of time (infusion) or bolus injections. The compounds/compositions described herein may also be administered by inhalation (e.g. intranasally), orally, transdermally or by the rectal or vaginal routes in certain circumstances. The compounds/compositions of described herein may also be incorporated into a slow or delayed release Formulation or device. Such Formulations or devices may, for example, be inserted on or under the skin and the compounds/compositions may be released over weeks or even months.

It will be appreciated that the amount of the compounds/compositions described herein required is determined by biological activity and bioavailability which in turn depends on the mode of administration, the physicochemical properties of the compound employed and whether the compound is being used as a monotherapy or in a combined therapy. The frequency of administration will also be influenced by the above mentioned factors and particularly the half-life of the compounds/compositions within the subject being treated.

Optimal dosages of the compounds/compositions to be administered may be determined by those skilled in the art, and will vary with the particular compound being used, the strength of the preparation, the mode of administration, and the advancement of the disease condition.

Additional factors depending on the particular subject being treated will result in a need to adjust dosages, including subject age, weight, gender, diet, and time of administration.

In using one or more of the compounds described herein for therapeutic or prophylactic purposes it will generally be administered so that a daily dose in the range, for example, a daily dose selected from 0.1 mg/kg to 100 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Suitably one or more of the compounds described herein may be administered orally, for example in the form of a tablet, or capsule dosage form. The daily dose administered orally may be, for example a total daily dose selected from 1 mg to 2000 mg, 5 mg to 2000 mg, 5 mg to 1500 mg, 10 g to 750 mg or 25 mg to 500 mg. Typically, unit dosage forms will contain about 0.5 mg to 0.5 g of one or more of the compounds described herein.

Known procedures, such as those conventionally employed by the pharmaceutical industry (e.g. in vivo experimentation, clinical trials, etc.), may be used to establish specific Formulations of the compounds/compositions as well as precise dosage regimens.

### Testing

The present methods may comprise determining the level of Galectin-3 or expression of LGALS3 or level of LGALS3 expression in a biological sample from the subject. Said subject may be suspected of having a disease, disorder, state or condition associated with expression or overexpression of Galectin-3. This may be before any administration and the method may then comprise administering a composition comprising the compound of Formula X and/or a composition comprising the compound of Formula Y, and/or a composition comprising the compound of Formula Z, or a pharmaceutically acceptable salt thereof, or a composition of one of these compounds, or one or more of the other compounds or compositions described herein, optionally with a pharmaceutically acceptable excipient, to said subject.

A test to determine Galectin-3 or expression of LGALS3 or a level of LGALS3 expression or overexpression of these may be performed on a biological sample, preferably prior to a subject being given a Galectin-3 inhibitor such as the compounds of Formula X, Formula Y or Formula Z. Such a test may inform a clinical practitioner whether the subject will respond to treatment with a Galectin-3 inhibitor, allowing better targeting of therapy. Suitably the Galectin-3 inhibitor is a compound described herein, for example one or more of the compounds of Formula X, Formula Y or Formula Z, or one of the other compounds as described herein.

The level or amount or expression may be based upon a measure of one type of target molecule indicative of this (i.e. measure of DNA, RNA or protein). The level may alternatively be based upon a measure of a combination of two or more types of target molecule indicative of expression (i.e. two or more of DNA, RNA and protein). The level of a target molecule may be expressed as a direct measure of the amount of target (for example concentration (mg/vol sample)).

The level of Galectin-3 or expression of LGALS3 or a level of LGALS3 expression may be assessed by any suitable method known in the art, for example, ELISA, Immunohistochemistry, Immunoblotting, mass spectrometry, flow cytometry, transcriptome analysis for determining biomarker expression. Suitable techniques for determining the level of RNA/mRNA in a sample, for example by transcriptome analysis, may include hybridization techniques, for example by detecting binding to a nucleic acid library, quantitative PCR, and high throughput sequencing including tag based sequencing such as SAGE (serial analysis of gene expression) and RNA-seq.

Predicting response by testing for Galectin-3 or expression of LGALS3 or a level of LGALS3 expression may increase the likelihood of treatment working, and it may be made while a subject is receiving alternative treatment, or before or after already planned alternative treatment.

Increased levels may be an increased amount or increased expression or overexpression compared to the level of the same target molecule in another subject (i.e. a subject with a normal level of Galectin-3 or expression of LGALS3 or a level of LGALS3 expression, or a subject known to not have a Galectin-3 related disorder, disease, state or condition), or compared to the level of the same target molecule in the same subject prior to them having the increased amount or increased expression, or before they were known to have a Galectin-3 related disorder, disease, state or condition. The level may be compared to a subject without a Galectin-3 related disorder, disease, state or condition or the literature values for subjects known to not have a Galectin-3 related disorder, disease, state or condition. These may in addition be compared to known reference or baseline values or compared to control samples, for example those from the literature or samples from subject without a Galectin-3 related disorder, disease, state or condition. These may be averages of many control samples or literature values. The levels may be in specific tissues or sample types. A control sample may be age matched with the patient undergoing investigation. Reference values or base line value may be obtained from suitable individuals and used as a general reference value for multiple analysis. Said subject may be suspected of having a disease, disorder, state or condition associated with expression or overexpression of Galectin-3.

Reference herein to a “biological sample” or “biological sample taken from a subject” or “sample” may be understood to be a reference to any sample of biological material derived from a subject such as, but not limited to, tumour tissue, suspected tumour tissue, a tissue biopsy, mucus, stool, tear, urine, blood, serum, cell extract, biopsy specimens and/or fluid which has been introduced into the body of an individual and subsequently removed such as, for example, a solution added to and then extracted from a tissue, for example the lung, or colon, for example an enema. The biological sample may be used directly, or may undergo alteration, treatment or change prior to use. A sample may be taken from a subject suspected of having a disease, disorder, state or condition associated with expression or overexpression of Galectin-3, or taken from a subject known to have a disease, disorder, state or condition associated with expression or overexpression of Galectin-3. The sample may be a blood, tumour, tissue biopsy, or suspected tumour sample. This may be prior to or sequentially with the herein described treatments with Galectin-3 inhibitors. This may include determining said level of or expression in a biological sample, for example a fluid (e.g. blood, tear or urine) sample or a tissue sample (e.g. a tissue biopsy, biopsy, tumour sample, suspected tumour sample) take from a subject. Said subject may be suspected of having a disease, disorder, state or condition associated with expression of Galectin-3.

Administration may be when any Galectin-3 or any expression of LGALS3 in the biological sample from said subject is found or determined. This may be when the level of Galectin-3 or expression of LGALS3 or level of LGALS3 expression in the biological sample from said subject is found to be higher than a base line or overexpressed. Higher than a base line or overexpressed may be compared to a reference, for example a literature reference or a reference sample, as described herein. Optionally, the method may further comprise the step of administering a composition comprising the compound of Formula X and/or a composition comprising the compound of Formula Y, and/or the compound of Formula Z, or a pharmaceutically acceptable salt thereof, optionally with a pharmaceutically acceptable excipient, to said subject when the level of Galectin-3 or expression of LGALS3 or level of LGALS3 expression in the biological sample from said subject is found to be higher than a base line or overexpressed, for example greater than a reference, for example a literature reference or a reference sample.

Accordingly, any method of treatment of the present invention may first involve diagnosing the subject has having a disease, disorder, state or condition associated with Galectin-3 or expression of LGALS3 or a level of LGALS3 expression greater than expected.

### Mechanism of Action

As previously stated, and without wishing to be bound by any particular theory, it is believed that the compounds described herein exert their therapeutic effects, at least in part, by inhibiting the activity of Galectin-3.

According to an aspect of the present invention, there is provided a compound of Formula X and/or Formula Y and/or Formula Z, and/or pharmaceutically acceptable salts thereof, or a composition of one of these compounds, or one or more of the other compounds or compositions described herein, for use in inhibiting Galectin-3 activity. This may be a composition comprising the same.

This may be for use in the treatment of a disease, disorder, state or condition where inhibiting Galectin-3 activity would be beneficial for the treatment of the disease, disorder, state or condition.

In a further aspect, the present invention provides the use of a compound of Formula X and/or Formula Y and/or Formula Z, and/or pharmaceutically acceptable salts thereof, or a composition of one of these compounds, or one or more of the other compounds or compositions described herein as an inhibitor of Galectin-3 activity (in vitro).

In a further aspect, the present invention provides a method of inhibiting the activity of Galectin-3 (in vitro or in vivo), the method comprising administering an effective amount of a compound of Formula X and/or Formula Y and/or Formula Z, and/or pharmaceutically acceptable salts thereof, or a composition of one of these compounds, or one or more of the other compounds or compositions described herein.

Galectin-3 binding compounds are known to inhibit the interaction between Galectin-3 and transmembrane mucin proteins such as MUC1 and MUC16 and solvate also to inhibit Galectin-3-induced cancer cell adhesion to the endothelial cells. This may play a part in the usefulness of the compound described herein for the treatment of cancer metastasis.

Thus, the present invention also provides a compound of Formula X and/or Formula Y and/or Formula Z, and/or pharmaceutically acceptable salts thereof, or a composition of one of these compounds, or one or more of the other compounds or compositions described herein, for use in the inhibition of Galectin-3 mediated binding of cancer cells to endothelial cells. Also, the use of a compound of Formula X or Formula Y or Formula Z, as defined herein, or a salt thereof, or another compound as described herein, as an inhibitor of Galectin-3 mediated binding of cancer cells to endothelial cells. Also, a method of inhibiting the Galectin-3 mediated binding of cancer cells to endothelial cells (in vitro or in vivo) by administering a compound of Formula X or Formula Y or Formula Z, as defined herein, or a salt thereof, or another compound as described herein. Also, a compound of Formula X or Formula Y or Formula Z, as defined herein, or a salt thereof, or another compound as described herein, for use in the inhibition of the interaction between Galectin-3 and its binding ligands (e.g. mucin proteins, growth factors, growth factor receptors, adhesion molecules, checkpoint molecules, other cell surface and intracellular molecules, matrix molecules). Also, the use of one or more of the compounds of Formula X or Formula Y or Formula Z, as defined herein, or a salt thereof, or another compound as described herein (i.e. the compound of Formula W), as an inhibitor of the interaction between Galectin-3 and its binding ligands intracellularly and extracellularly. Also, a method of inhibiting the activity of Galectin-3 binding to cancer-associated cell membrane proteins (in vitro or in vivo), the method comprising administering an effective amount of a compound of Formula X or Formula Y or Formula Z, as defined herein, or a salt thereof, or another compound as described herein.

### EXAMPLES

The invention will now be illustrated in the following Examples.

### Example 1—Compounds of Formula X, Formula Y and Formula Z Inhibit Galectin-3 Binding to its Ligand Asialofetuin (ASF)

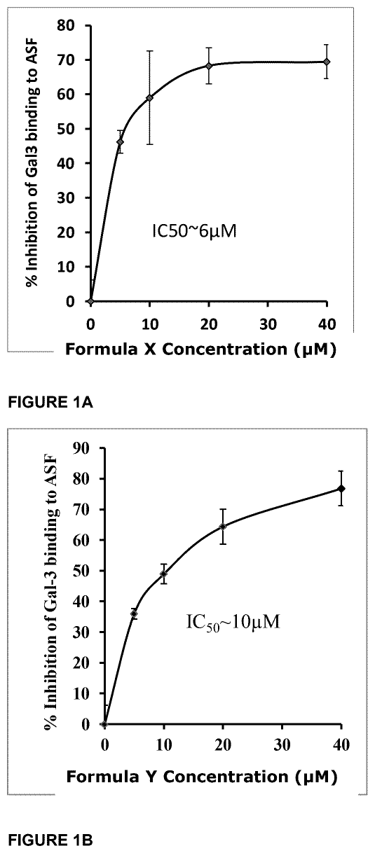

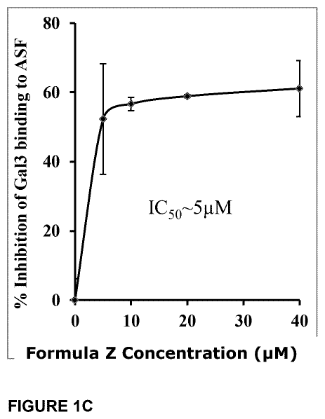

FIG. 1A, FIG. 1B and FIG. 1C show compounds of Formula X, Formula Y and Formula Z inhibit Galectin-3 binding to its ligand asialofetuin (ASF). Binding of Galectin-3 (5 μg/ml) to asialofetuin (ASF, 20 μg/ml) in the absence or presence of different concentrations of the compound of Formula X (FIG. 1 FIG<A), the compound of Formula Y (FIG. 1B) or the compound of Formula Z (FIG. 1C) was assessed by ELISA. Compounds of Formula X, Formula Y and Formula Z inhibit galectin-3 binding to ASF in a dose-dependent manner.

### Example 2—Determination of the compound of Formula X binding affinity to Galectin-3

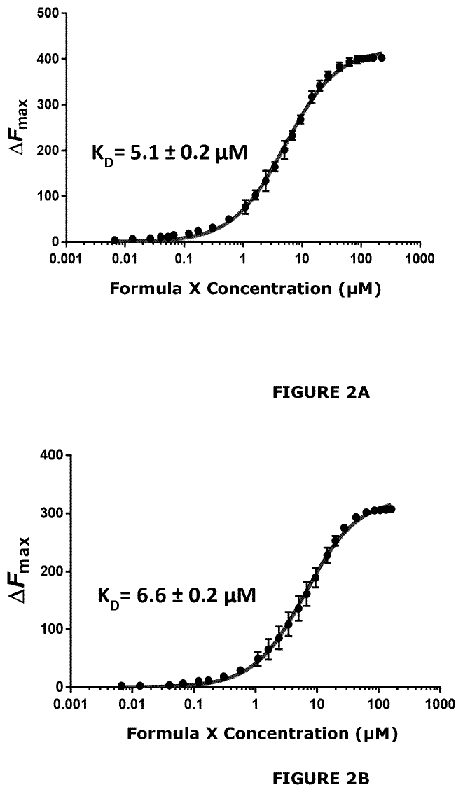

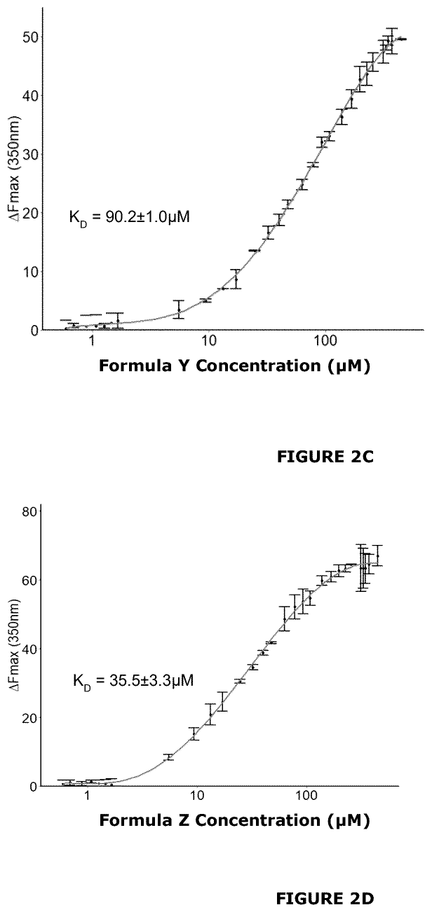

FIG. 2A, FIG. 2B, FIG. 2C and FIG. 2D show the analysis of the binding affinity of the compounds of Formula X, Formula Y and Formula Z to Galectin-3. The Binding of the compound of Formula X to full-length (FIG. 2A) and C-terminal (aa 115-250) Galectin-3 (FIG. 2B) is shown, along with the binding of Formula Y (FIG. 2C) and Formula Z (FIG. 2D) to Galectin-3. These were assessed by Tryptophan Fluorescence Spectroscopy (TFS). The compound of Formula X binds to the full-length and C-terminal galectin-3 with binding affinity (KD) at 5~7 μM. The compound of Formula Y binds to the full-length galectin-3 with binding affinity (KD) at ~90 μM. The compound of Formula Z binds to the full-length galectin-3 with binding affinity (KD) at ~35 μM.

### Example 3—Inhibition of Cancer Cell Adhesion to Vascular Endothelial Cells

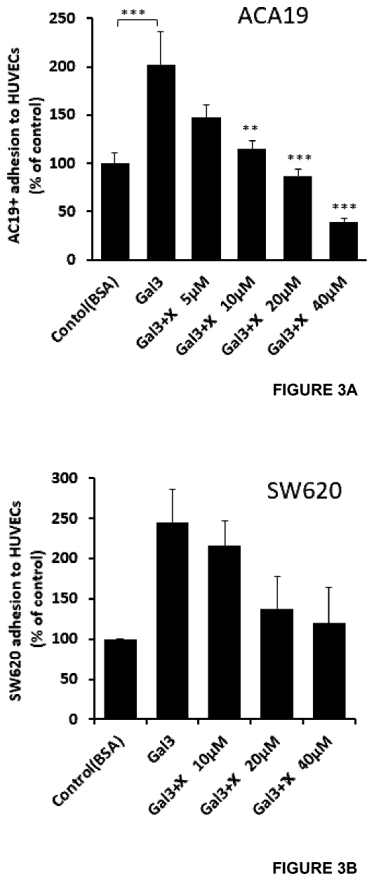

FIG. 3A and FIG. 3B show the compound of Formula X inhibits cancer cell adhesion to vascular endothelial cells. Adhesion of human melanoma ACA19 (FIG. 3A) and colon cancer SW620 (FIG. 3B) cells to human umbilical vein endothelial cells (HUVECs) was assessed in the absence or presence of Galectin-3 (2 μg/ml) and various concentrations of the compound of Formula X. The compound of Formula X causes dose-dependent inhibition of Galectin-3-mediated adhesion of SW620 and ACA19 cells to HUVECs. ***P<0.001, **p<0.01, *p<0.05 (ANOVA).

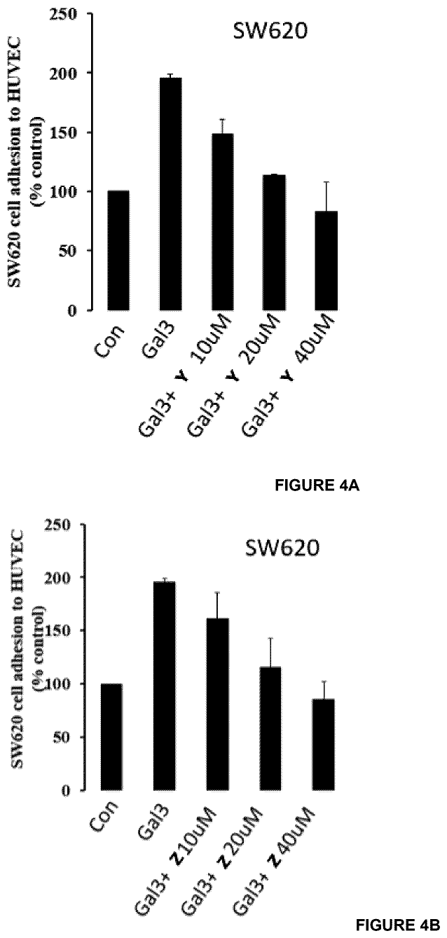

FIG. 4A and FIG. 4B show the compound of Formula Y and the compound of Formula Z inhibit cancer cell adhesion to vascular endothelial cells. Adhesion of human colon cancer SW620 cells to human umbilical vein endothelial cells (HUVECs) was assessed in the absence or presence of Galectin-3 (2 ug/ml) and various concentrations of the compound of Formula Y (FIG. 4A) or the compound of Formula Z (FIG. 4B). Both compounds cause dose-dependent inhibition of Galectin-3-induced SE620 cell adhesion to HUVECs.

### Example 4—Determination of Cancer Cell Invasion Through HUVECs

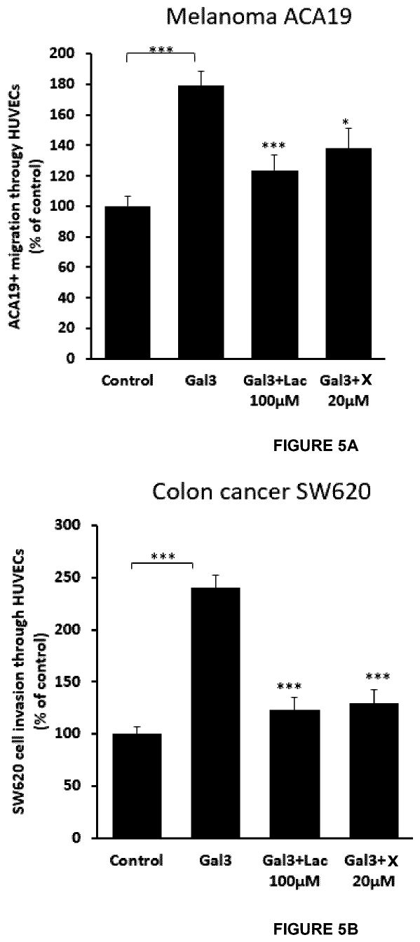

FIG. 5A and FIG. 5B show the compound of Formula X inhibits cancer cell invasion through vascular endothelial cells. Invasion of human melanoma ACA19 (FIG. 5A) and colon cancer SW620 (FIG. 5B) cells through human umbilical vein endothelial cells (HUVECs) was assessed in transwells in the absence or presence of the compound of Formula X (20 μM) or a Galectin-3 disaccharide inhibitor lactose (Lac, 100 μM). The compound of Formula X causes significant inhibition of Galectin-3-mediated invasion of ACA19 and SW620 cells through HUVECs. ***P<0.001, *p<0.05 (ANOVA).

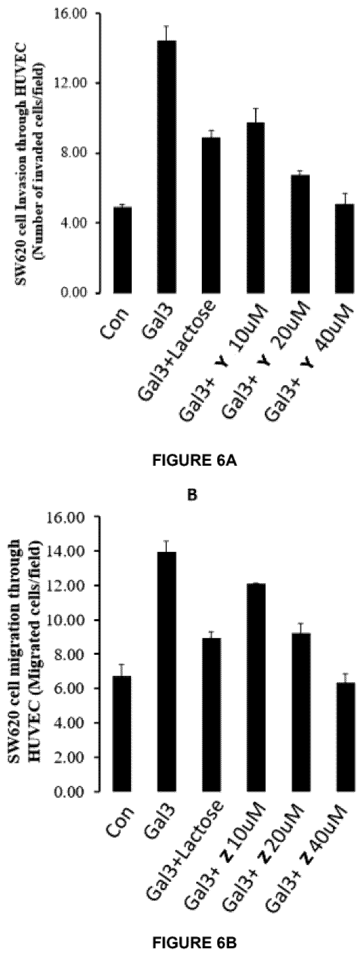

FIG. 6A and FIG. 6B show the compound of Formula Y and the compound of Formula Z inhibit cancer cell invasion through vascular endothelial cells. Invasion of human colon cancer SW620 cells through human umbilical vein endothelial cells (HUVECs) was assessed in transwells in the absence or presence of Galectin-3 (1 ug/ml), a galectin-3 disaccharide inhibitor lactose (100 uM), the compound of Formula Y and the compound of Formula Z. The compound of Formula Y (FIG. 6A) and the compound of Formula Y (FIG. 6B) both cause dose-dependent inhibition of galectin-3-mediated invasion of SW620 cells through HUVECs, similarly as the Galectin-3 inhibitor lactose.

### Example 6—Inhibition of Vascular Cell Tubule Formation (Angiogenesis)

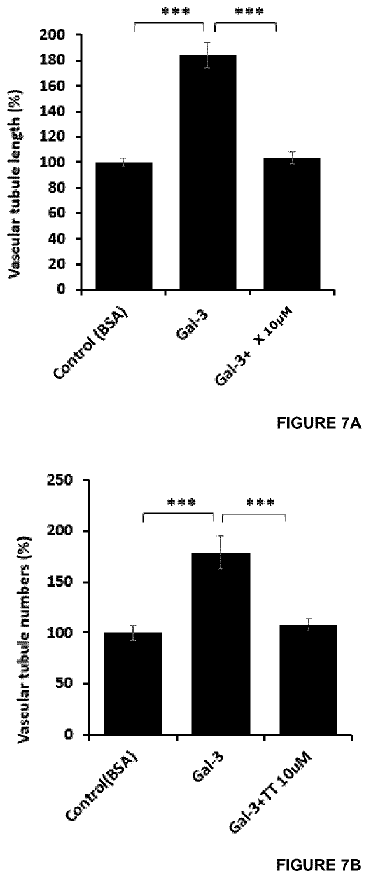

FIG. 7A and FIG. 7B the compound of Formula X inhibits vascular cell tubule formation (angiogenesis). Vascular cell tubule formation of human umbilical vein endothelial cells (HUVECs) was assessed in the absence or presence of Galectin-3 (2 μg/ml) and the compound of Formula X (10 μM). The presence of the compound of Formula X causes significant inhibition of galectin-3-induced vascular cell tubule formation when vascular tubule length (FIG. 7A) and tubule numbers (FIG. 7B) were measured. ***P<0.001 (ANOVA).

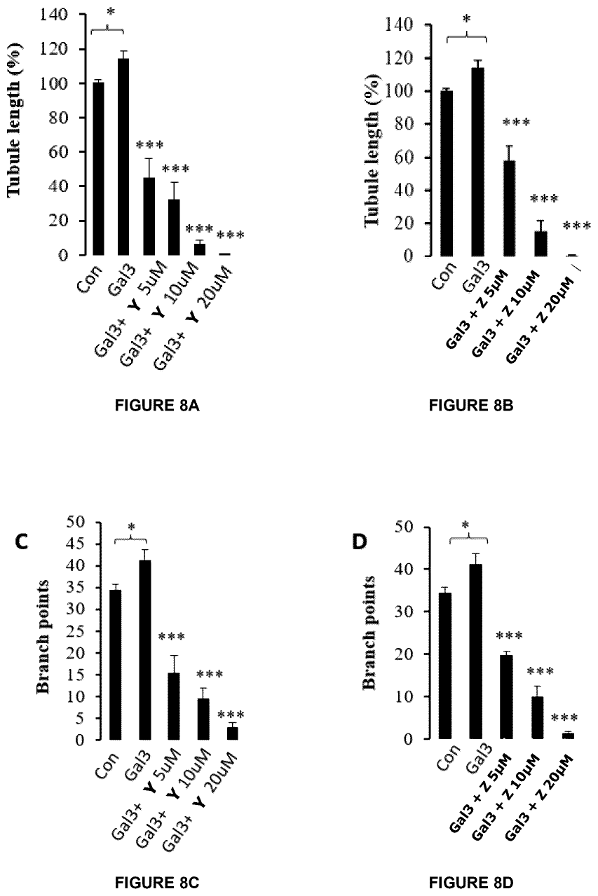

FIG. 8A, FIG. 8B, FIG. 8C and FIG. 8D show the compound of Formula Y and the compound of Formula Z inhibit vascular cell tubule formation (angiogenesis). Vascular cell tubule formation of human umbilical vein endothelial cells (HUVECs) was assessed in the absence or presence of Galectin-3 (2 μg/ml), various concentrations of the compound of Formula Y (FIG. 8A and FIG. 8C) or the compound of Formula Z (FIG. 8B and FIG. 8D). The presence of the compound of Formula Y or the compound of Formula Z causes dose-dependent inhibition of vascular cell tubule formation when vascular tubule length (FIG. 8A and FIG. 8B) and tubule numbers (FIG. 8C and FIG. 8D) were measured. *P<0.05, *** P<0.001 (ANOVA).

### Example 7—Determination of the Compound Cytotoxicity

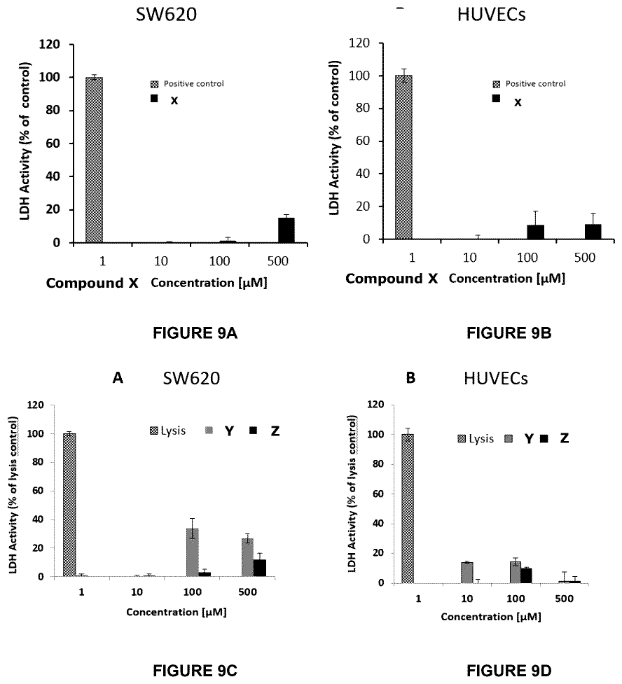

FIG. 9A, FIG. 9B, FIG. 9C and FIG. 9D show the compounds of Formula X, Formula Y and Formula Z exhibit little or low toxicity. Cytotoxicity of the compound of Formula X to human colon cancer SW620 (FIG. 9A) and vascular HUVECs (FIG. 9B) cells was assessed in the absence or presence of various concentrations of the compound of Formula X by Lactate dehydrogenase (LDH) release assay. The compound of Formula X shows no detectable cytotoxicity to both human HUVEC cells and SW620 cells at 100 μM. Cytotoxicity of the compounds of Formula Y and Formula Z to human colon cancer SW620 (FIG. 9C) and vascular HUVECs (FIG. 9D) cells was assessed in the absence or presence of various concentrations of the compounds of Formula Y and Formula Z by Lactate dehydrogenase (LDH) release assay. The compounds of Formula X and Formula Z show low cytotoxicity to both human HUVEC cells and SW620 cells.

### Example 8—Determination of the Compound of Formula X Selectivity

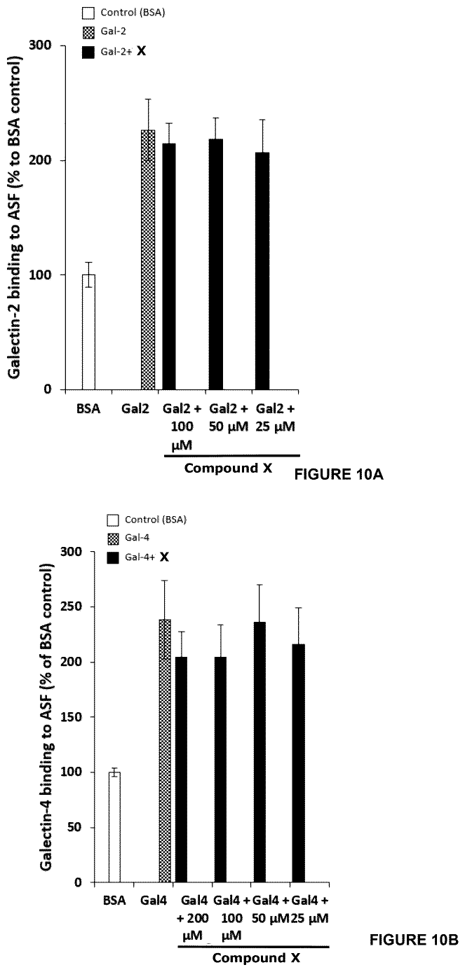

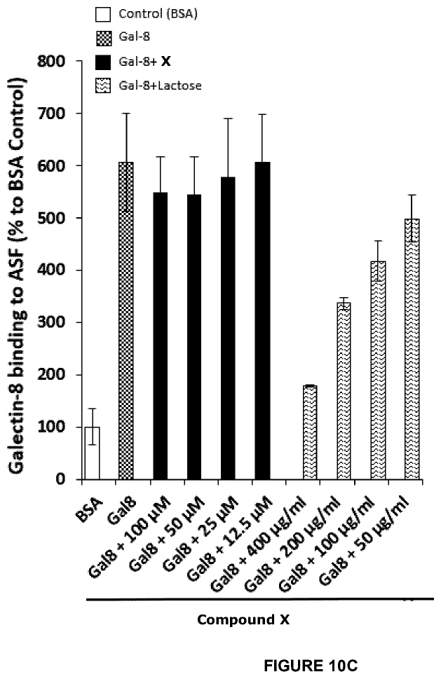

FIG. 10A, FIG. 10B and FIG. 10C show the compound of Formula X does not affect binding of galectins-2, -4 and -8 to ligand asialofetuin. Binding of Galectins-2 (Gal-2, FIG. 10A), -4 (Gal-4, FIG. 10B) and -8 (Gal-8, FIG. 10C) (5 μg/ml) to asialofetuin (ASF, 20 μg/ml) in the absence or presence of different concentrations of the compound of Formula X or positive control galectin inhibitor lactose was assessed by ELISA. The compound of Formula X does not affect binding of these galectin members to ASF. The disaccharide lactose dose-dependently inhibits Galectin-8 binding to ASF (positive control) (FIG. 10C).

### Example 9—Determination of Secretion of Pro-Inflammatory Cytokine from Macrophages

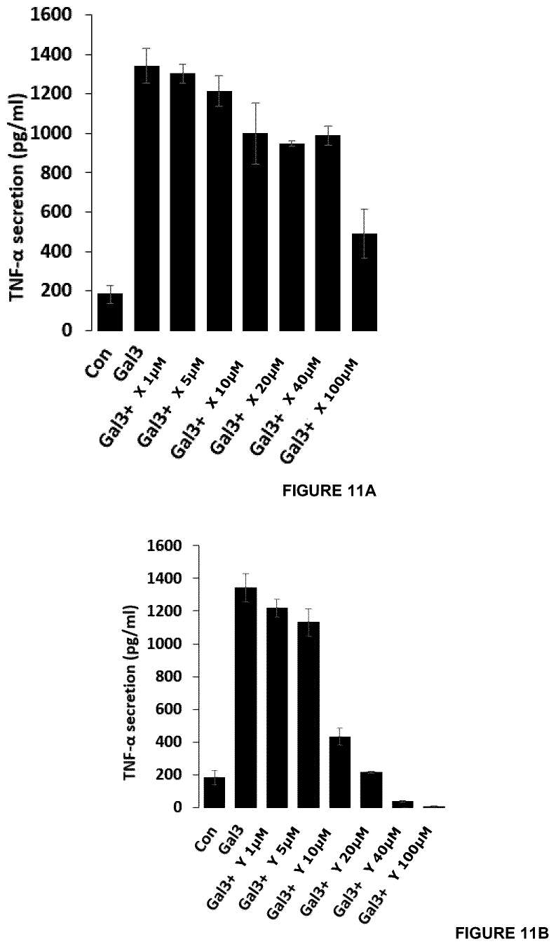

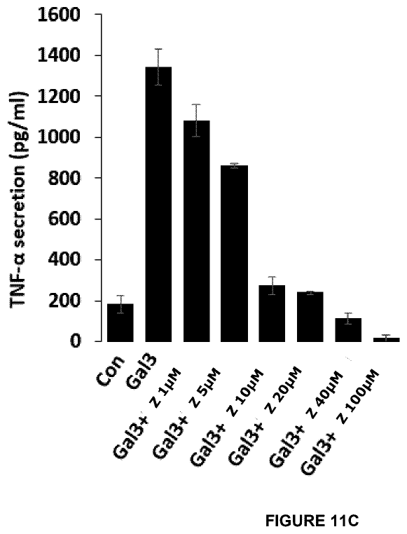

FIG. 11A, FIG. 11B and FIG. 11C. show the compounds of Formula X, Formula Y and Formula Z inhibit galectin-3-induced secretion of pro-inflammatory cytokine TNFα from macrophages. Secretion of TNFα from THP-1 differentiated macrophages was analysed in the absence or presence of Galectin-3 (10 μg/ml) and various concentrations of Formula X (FIG. 11A), Formula Y (FIG. 11B) or Formula Z (FIG. 11C) by ELISA. Galectin-3 markedly increases secretion of TNFα and the presence of the compounds of Formula X, Formula Y and Formula Z causes dose-dependent inhibition of galectin-3-induced TNFα secretion.

FIG. 12A, FIG. 12B and FIG. 12C. show the compounds of Formula X, Formula Y and Formula Z inhibit galectin-3-induced secretion of pro-inflammatory cytokine IL-1β from macrophages. Secretion of IL-1β from THP-1 differentiated macrophages was analysed in the absence or presence of Galectin-3 (10 μg/ml) and various concentrations of Formula X (FIG. 12A), Formula Y (FIG. 12B) or Formula Z (FIG. 12C) by ELISA. Galectin-3 markedly increases secretion of IL-1p and the presence of the compounds of Formula X, Formula Y and Formula Z causes dose-dependent inhibition of galectin-3-induced IL-13 secretion.

### Example 10—Inhibition of Human Lung Cancer Cell Adhesion to Basement Matrix

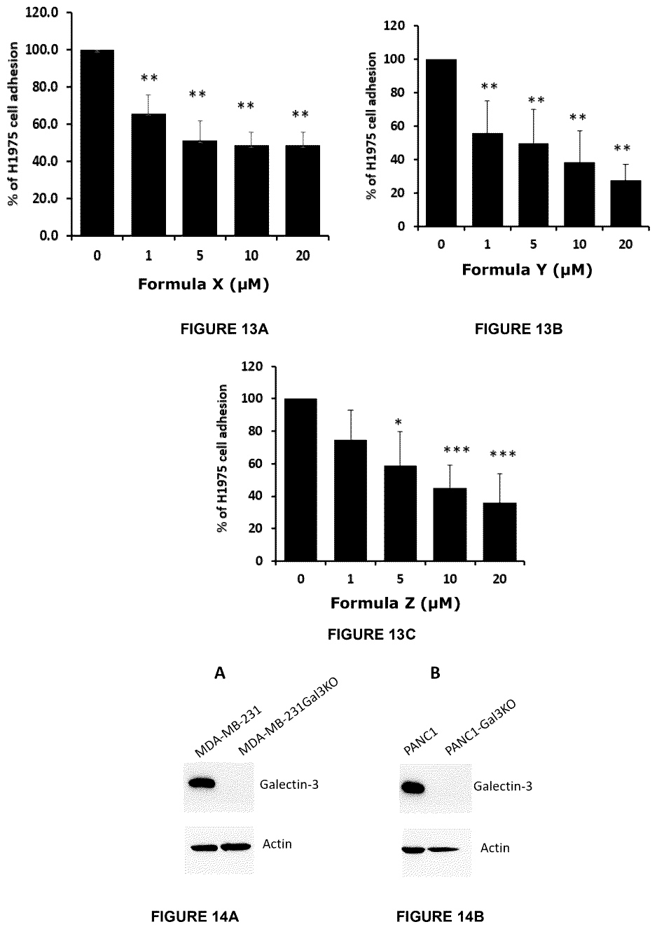

FIG. 13 shows inhibition of human lung cancer cell adhesion to basement matrix by the compound of Formula X (FIG. 13A), the compound of Formula Y (FIG. 13B), the compound of Formula Z (FIG. 13C), each causes dose-dependent inhibition of human lung cancer H1975 cell adhesion to matrix with IC₅₀~5 μM for the compounds of Formula X and Y and ~5~10 μM for the compound of Formula Z respectively **p<0.01, ***p<0.001.

### Example 11—Galectin-3 Knockout of Human Triple-Negative Breast Cancer MDA-MB-231 (A) and Pancreatic Cancer PANC1 (B) Cells by CRISPR/Cas9

FIG. 14 shows Galectin-3 Knockout of human triple-negative breast cancer MDA-MB-231 (A) and pancreatic cancer PANC1 (B) cells by CRISPR/Cas9. The galectin-3 expression in the control and knockout cells were determined by galectin-3 immunoblotting. The same blots were probed by anti-actin antibody for verification of protein loading.

### Example 12—Inhibition of Galectin-3-Mediated Human Triple-Negative Breast Cancer Cell Adhesion to Basement Matrix

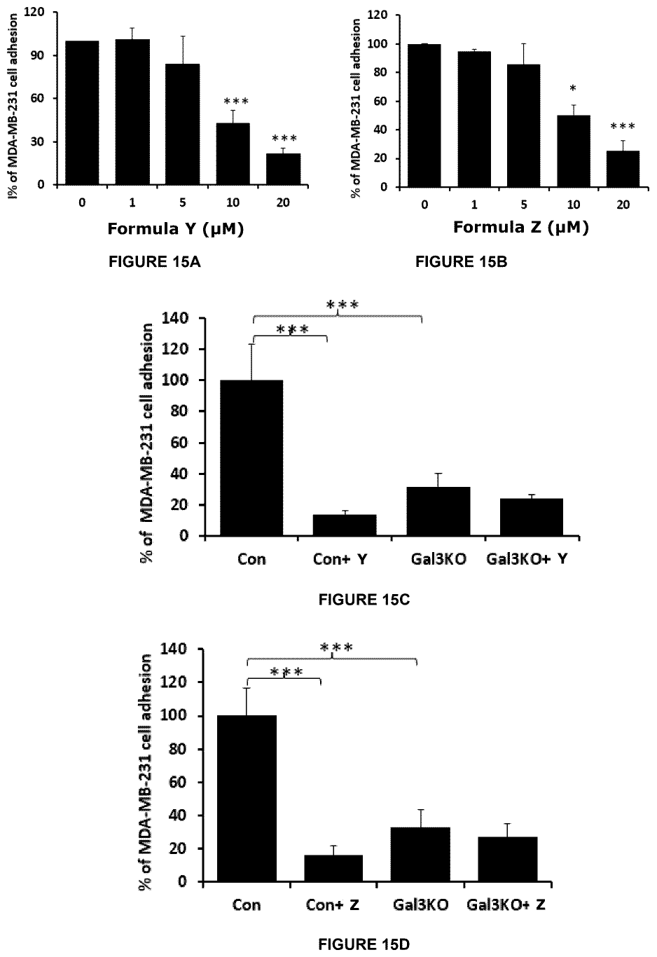

FIG. 15 shows Inhibition of galectin-3-mediated human triple-negative breast cancer cell adhesion to basement matrix by the compound of Formula Y and the compound of Formula Z. The compound of Formula Y (FIG. 15A) and the compound of Formula Z (FIG. 15B) each causes dose-dependent inhibition of triple-negative breast cancer MDA-MB-231 cell adhesion to matrix, with IC₅₀~10 μM for the compounds of Formula X and Y. In comparison to the control cells, galectin-3 knockout resulted in significant reduction of MDA-MB-231 cell adhesion to matrix. The presence of the compound of Formula Y (FIG. 15C) and the compound of Formula Z (FIG. 15D) both caused significant reduction of the non-transfected (con) but not the galectin-3 knockout (Gal3KO) MDA-MB-231 cells ***p<0.05, p<0.01, ***p<0.001.

### Example 13—Inhibition of Galectin-3-Mediated Adhesion of Human Pancreatic Cancer Cells to Basement Matrix

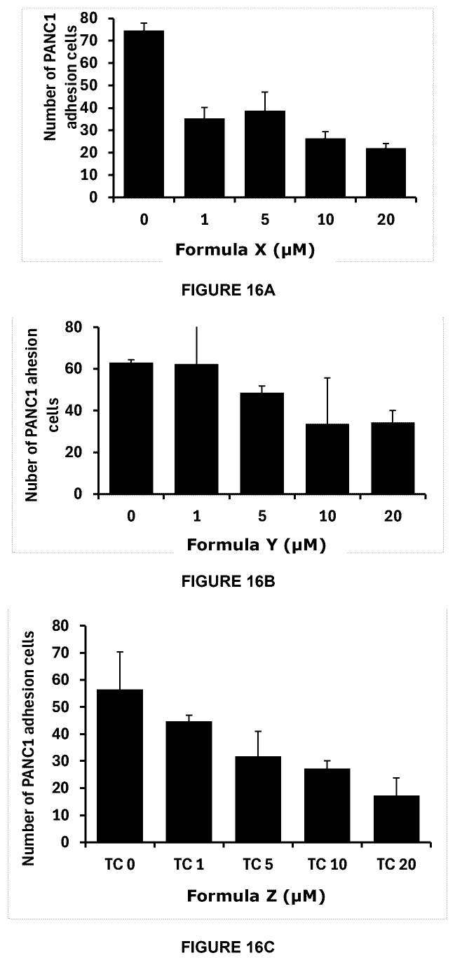

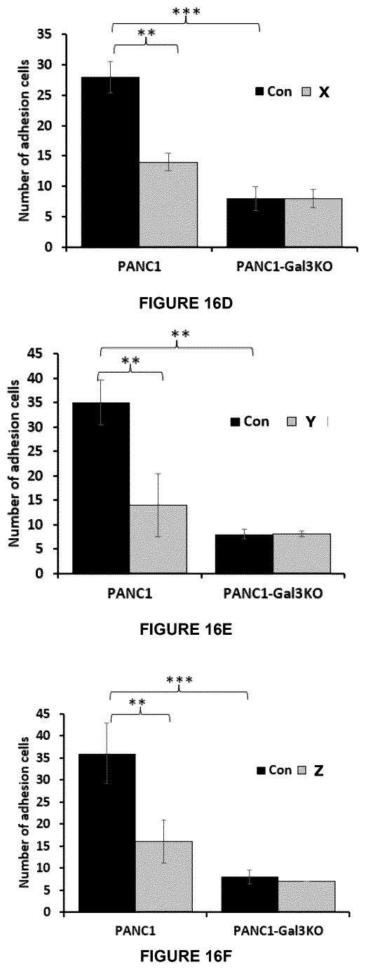

FIG. 16 shows Inhibition of galectin-3-mediated adhesion of human pancreatic cancer cells to basement matrix by the compounds of Formula X, Y and Z. The compound of Formula X (FIG. 16A), the compound of Formula Y (FIG. 16B), the compound of Formula Z (FIG. 16C), each causes dose-dependent inhibition of human pancreatic cancer PANC1 cell adhesion to matrix. In comparison to the control cells, galectin-3 knockout resulted in significant reduction of PANC1 cell adhesion to matrix. The presence of the compound of Formula X (FIG. 16D), the compound of Formula Y (FIG. 16E) and the compound of Formula Z (FIG. 16F) all caused significant reduction of the non-transfected (con but not the galectin-3 knockout (Gal3KO) PANC1 cells. *p<0.05, **p<0.01, ***p<0.001.

### Materials

Monoclonal antibodies to galectins-2, -3, -4 or -8 antibodies, human recombinant galectin-8 were obtained from R&D Systems (Abingdon, UK). HRP-conjugated anti-mouse antibody was obtained from Dako (Glostrup, Denmark). FAST OPD tablets and asialo fetuin (ASF) were obtained from Sigma-Aldrich (Dorset, UK). Falcon 8 μm pore trans-well inserts and LDH cytotoxicity kits were from Thermo Fisher Scientific (Waltham, USA). Matrigel Matrix was purchased from BD Biosciences (Wokingham, UK). Fluorescent cell labelling solution DiL was obtained from Molecular Probes (Eugene, OR). Thermo Fisher. HUVECs, EBM and EBM-2 Bullet kits were obtained from Lonza (Verviers, Belgium). Human recombinant galectins-2, -3 and -4 were produced in E. Coli as described [Sindrewicz, P., et al., Intrinsic tryptophan fluorescence spectroscopy reliably determines galectin-ligand interactions. Sci Rep, 2019. 9(1): p. 11851]. CRISPR/Cas9 galectin-3 knockout plasmid (sc-417680), galectin-3 HDR plasmid (sc-417680-HD) and Plasmid Transfection Medium (sc-108062) were obtained from Santa Cruz.

### Determination of Galectin Binding to its Ligand

Effect of the inhibitors on galectin binding to its ligand asialo fetuin (ASF) was measured by galectin ELISA. Nanty-six well plates were coated overnight with 20 μg/ml ASF in coating buffer (15 mM Na2CO3 17 mM NaHCO₃ pH 9.6.). The wells were washed twice with PBS and incubated with blocking buffer (1% BSA in PBS) for 1 hr. The supernatants were removed and replaced with 5 μg/ml recombinant human galectin without or with different concentrations of the compound inhibitors or lactose in blocking buffer for 1 hr at room temperature. The wells were washed and incubated with 1 μg/ml anti-galectin antibody for 2 hr. Following addition of HRP-conjugated secondary antibody for 1 hr, FAST OPD development solution was added for 15-20 minutes. The reaction was terminated by addition of 4M sulphuric acid. The plates were read at 492 nm (reference wavelength 595 nm) by a microplate reader.

### Determination of Compound Binding Affinity to Galectin-3

The binding affinity of the inhibitors to galectin-3 was determined by Intrinsic Tryptophan Fluorescence Spectroscopy (TFS) exactly as we described recently [Sindrewicz, P., et al., Intrinsic tryptophan fluorescence spectroscopy reliably determines galectin-ligand interactions. Sci Rep, 2019. 9(1): p. 11851]. The Galectin-3 concentration used was 10 μM and the inhibitor titration concentrations were from 0 to 224 μM. DMSO/control buffer (PBS with 2 mM EDTA) titrations were performed and used for background subtraction.

### Determination of Cancer Cell Adhesion to HUVECs

HUVEC cells at 1×10⁵ cells/ml in EGM medium were seeded into 24 well plates pre-inserted with 13 mm glass coverslip. The cells were incubated at 37° C. at 5% CO₂ for 24 hr for monolayer formation. AGA19+ or SW620 cell suspension (5×10⁵ cells/ml in DMEM) was introduced with 5 μl/ml fluorescent cell labelling solution DiL for 30 min at 37° C. The fluorescent labelled cells were washed twice with PBS and resuspended into 1×10⁵ cells/ml in DMEM. The cancer cell suspension was then introduced with galectin-3 (2 μg/ml) and various concentrations of the compound inhibitors at 37° C. for 1 hr. The wells were carefully washed twice with PBS to remove the non-adhesion cells before the cells in the wells were fixed with 2% paraformaldehyde for 10 min. The glass coverslip was carefully removed from the plate, placed on a glass slide. After addition of fluorescence mounting medium containing DAPI, fluorescence labelled cells were quantified under a fluorescence microscope.

### Determination of Cancer Cell Invasion Through HUVECs

HUVECs were plated at a density of 1.5×10⁴ cells in 8 μm pore transwells in 24-well plate and cultured for three days to allow formation of tight monolayer. The integrity of HUVEC monolayer was confirmed by measuring TEER and only monolayers with TEER reading>800 Ω/cm² were used for the invasion assay. AGA19+ or SW620 cell suspension (5×10⁵ cells/ml in DMEM) was introduced with 5 μl/ml fluorescent cell labelling solution DiL for 30 min at 37° C. The fluorescence labelled cells were washed twice with PBS and resuspended into 1×10⁵ cells/ml in DMEM. The cancer cell suspension was then incubated with or without galectin-3 (2 μg/ml) or various concentrations of the compound inhibitors or lactose at 37° C. for 20 min before introduction to the HUVEC monolayers for 24 hr at 37° C. The cells at the upper side of the trans-well inserts were gently removed with cotton swabs. The cells migrated to the bottom side of the transwells were fixed with 2% paraformaldehyde for 10 min. The trans-well membrane was removed and placed on glass slides. After addition of fluorescence mounting medium containing DAPI, the fluorescence labelled cells on the membrane were quantified under a fluorescence microscope.

### Determination of the Inhibitors on Angiogenesis

Matrigel matrix (50 μl) was added to 96-well plates for 60 min at 37° C. to allow gel formation. HUVEC cells (1×10⁵ cells/ml in 1:1 EGM/EBM2 media) was introduced to the plates without or with galectin-3 (2 μg/ml) or different concentrations of compound inhibitors for 24 hr at 37° C. Endothelial cell tube formation was examined with Laser Microscope. The total number of branching points and the sum of the lengths of tubes were measured.

Determination of the Compound Cytotoxicity

SW620 cells (5×10⁴ cells/ml) were seeded into 96-well plate in DMEM (and incubated at 37° C. 5% CO₂ for 24 hr. HUVECs (1.0×10⁵ cells/ml) was seeded in EBM medium and incubated for 24-36 hr until they formed a monolayer. Compound inhibitors at different concentrations were then introduced to the cells (10× lysis buffer was included as maximum LDH activity control) for 24 hr. The culture medium was transferred to a new plate and was mixed with equal volume of reaction mixture provided in the kit for 30 min at room temperature before the absorbance was measured at 490 nm (reference 680 nm) by a spectrophotometer.

Determination of Secretion of Pro-Inflammatory Cytokine from Macrophages

Effect of the compound inhibitors on secretion of pro-inflammatory cytokines from macrophages were assessed in THP-1 monocyte differentiated macrophages. THP-1 cells were induced to differentiation into macrophages by incubation with 10 ng/ml PMA for 48 h at 37° C. The culture medium was then replaced with fresh medium without or with 10 μg/ml galectin-3 and various concentrations of the compound inhibitors for 24 h at 37° C. The culture medium was collected and the concentrations of TNFα and IL-1β in the culture medium were determined by TNFα and IL-1β ELISA.

### Determination of Cancer Cell Adhesion to Matrix Proteins

Nanty-six well plates were coated with 50 μl Matrigel (1:30 diluted in PBS) at 37° C. for 1 hour.

Cancer cells, pre-labelled with fluorescent die DiI at 37° C. for 20 minutes, were washed twice with PBS and resuspended into 3×10⁴ cells/ml in serum-free medium. The matrix-coated 96-well plates were washed twice with PBS before introduction of 100μ cell suspension with different concentrations of the inhibitors or PBS/DMSO (control) for 1 hour at 37° C. The plates were washed twice with PBS before three randomly selected Field of Views (FOVs) were recorded by fluorescence microscope and number of adhesion cells were quantified by Image J.

### Galectin-3 Knockout by Crispr/Cas9

MDAMB-231 or PANC-1 cells were seeded into 6-well plate (2×10⁵ cells) and cultured at 37° C. for 24 hour. Galectin-3 CRISPR Cas9 Knockout plasmid (2 μg) and galectin-3 HDR plasmid (2 μg) was mixed with Plasmid Transfection Medium (21 μl) for 5 minutes before addition to a mixture of lipofectamine STEM Transfection Reagent (STEM00001) (1 μl) and Plasmid Transfection Medium (25 μl) for 10 minutes at room temperature. The cells in the 6-well plates were washed and introduced with the Plasmid DNA/Lipofectamine STEM Transfection Reagent complex at 37° C. for 24 hour. The culture medium was replaced with fresh antibiotics-free medium for further 24 hour before introduction of 10 μg/mL puromycin-containing medium every three days. Successful co-transfection of the CRISPR/Cas9 KO Plasmid and HDR Plasmid was confirmed by expression of red fluorescence protein of the cells. Galectin-3 knockout in the cells was further confirmed by immunoblotting.

It will be clear to one skilled in the art that many improvements and modifications can be made to the foregoing exemplary embodiments without departing from the scope of the present disclosure.

1. (canceled)

2. A method of treating or preventing a disease, disorder, state or condition in a subject for which inhibiting Galectin-3 would be beneficial for the treatment and/or prevention of the disease, disorder, state or condition the method comprising: administering an effective amount of a compound of Formula X or a pharmaceutically acceptable salt thereof, and/or a compound of Formula Y or a pharmaceutically acceptable salt thereof, and/or a compound of Formula Z or a pharmaceutically acceptable salt thereof.

3. The method of claim 2, wherein the disease, disorder, state or condition is cancer, cancer metastasis, fibrosis, inflammation, diabetes and/or one or more other disease, disorder, state or condition affected and/or mediated by Galectin-3.

4. (canceled)

5. The method of claim 2, wherein the disease, disorder, state or condition is cancer and wherein the treatment and/or prevention of cancer comprises inhibiting or preventing metastasis, cancer reappearance after initial therapeutic treatment or intervention, cancer cell proliferation, tumour growth, angiogenesis, or immunosuppression.

6. The method of claim 2, wherein the disease, disorder, state or condition is cancer metastasis and wherein the treatment and/or prevention of cancer metastasis comprises inhibition of the motility of cancer or tumour cells, inhibition of angiogenesis, inhibition of immunosuppression, inhibition of the dissemination and invasiveness of cancer cells leading to inhibition of metastatic tumour growth, inhibitor of the dissemination and invasiveness of cancer cells leading to improved clinical outcome for the subject.

7. The method of claim 5, wherein the cancer is one or more of: breast cancer, triple negative breast cancer, pancreatic cancer, lung cancer, melanoma, colorectal cancer, head and neck cancer, ovarian cancer, oesophageal cancer, thyroid cancer, bladder cancer, urinary system cancer, prostate cancer, digestive or gastrointestinal tract cancer, leukaemia, lymphoma, liver cancer or musculoskeletal cancer.

8. The method of claim 2, wherein the disease, disorder, state or condition is fibrosis and wherein the treatment and/or prevention of fibrosis comprises treatment/prevention of a fibrotic disorder, a fibrotic condition, a fibroproliferative disease, or a disorder or state of wound-healing response that is out of control.

9. The method of claim 8, wherein the fibrotic disorder, fibrotic condition, fibroproliferative disease, or a disorder or state of wound-healing response that is out of control is one or more of: scarring or wound healing abnormalities, chronic or acute inflammation, chronic graft rejection, or a fibrotic condition or fibrosis affecting the heart, brain, lungs, liver, kidneys, heart or vascular system, mediastinum, bone, retroperitoneum, skin, digestive or gastrointestinal tract, connective tissue, eye or muscles.

10. The method of claim 2, wherein the disease, disorder, state or condition is inflammation and wherein the treatment and/or prevention of inflammation comprises treatment/prevention of an inflammation-related disease or a condition caused by inflammation.

11. The method of claim 10, wherein the inflammation-related disease or a condition caused by inflammation is one or more of: acute or chronic organ transplant rejection, graft-versus-host disease, inflammatory bowel disease, inflammatory skin disease, multiple sclerosis, arteriosclerosis, pancreatitis, acute bronchitis, chronic bronchitis, Alzheimer's disease, inflammatory lung disease, inflammatory skin disease, Acute bronchiolitis, folliculitis, chronic bronchiolitis, musculoskeletal pain or connective tissue inflammation, osteoarthritis, gout, spondyloarthropathy, Reiter's syndrome, psoriatic arthropathy, inflammation caused by bacterial, fungal, and viral infections one or more diseases, atherosclerosis, dermatitis, diverticulitis, fibromyalgia, hepatitis, irritable bowel syndrome, nephritis, Parkinson's disease, chronic inflammation, one or more neurodegenerative diseases or disorders caused by tumours caused by chronic inflammation and aging caused by chronic inflammation, inflammatory symptoms caused by autoimmune diseases including ulcerative colitis (UC) and Crohn's disease (CD), lupus erythematosus, hyperthyroidism, IgA nephritis, type I or type II diabetes and its complications, dry eye syndrome, rheumatoid arthritis, simple obesity, ankylosing spine inflammation, bronchial asthma, neurodermatitis, ulcerative colitis, oral ulcers, psoriasis, vitiligo, Behçet's disease, autoimmune iridocyclitis, autoimmune eczema, autoimmune uveitis, One or more of autoimmune conjunctivitis, autoimmune dry eye, autoimmune glaucoma, autoimmune cataract, allergic rhinitis, irritable bowel syndrome, pruritus.

12. The method of claim 2, wherein the disease, disorder, state or condition is diabetes and wherein the treatment and/or prevention of fibrosis comprises treatment/prevention of type I or type II diabetes and/or any related diabetic-associated disease or condition such as obesity.

13. The method of claim 2, wherein the disease, disorder, state or condition is Asthma, Atherosclerosis, Atopic Dermatitis, Cerebral infarction, COPD, Degenerative Aortic Stenosis, Endometriosis, Encephalitis, Gastritis, HIV infection, Interstitial lung disease, Juvenile Idiopathic Arthritis, CVD mortality, Non-alcoholic steatohepatitis (NASH), Obesity, Pneumonia, Pulmonary hypertension, Plaque Psoriasis, Q Fever, Rheumatoid Arthritis, Systemic Sclerosis, Urinary tract infections, Covid-19, sepsis, Thrombosis, Venous Thrombosis, Wound Healing, Cardiac syndrome X (CSX), Yeast infection-Candidiasis or Zoster-related pain (allodynia).

14. (canceled)

15. (canceled)

16. The method of claim 2, wherein the composition further comprises one or more other active agent, preferably one or more additional anti-tumour agents, and/or chemotherapy agents, and/or anti-metastatic agents, and/or radiotherapy agents and/or immunotherapeutic agents and/or anti-fibrosis/fibrotic agents and/or anti-inflammation/inflammatory agents and/or one or more other Galectin inhibitor.

17-29. (canceled)

30. The method of claim 2, wherein the composition is simultaneously or sequentially administered in combination with one or more other treatments, preferably wherein the one or more other treatments are one or more of chemotherapy, radiotherapy, immunotherapy, surgery, and/or surgical approaches to cancer treatments, preferably de-baulking or cryosurgery.

31. The method of claim 2, wherein the method further comprises determining the level of Galectin-3 or expression of LGALS3 or level of LGALS3 expression in the subject, and/or wherein the method further comprises determining the level of Galectin-3 or expression of LGALS3 or level of LGALS3 expression in a biological sample taken from the subject.

32. The method of claim 31, wherein said subject is suspected of having a disease, disorder, state or condition associated with expression of Galectin-3 or wherein said subject is known to have a disease, disorder, state or condition associated with expression of Galectin-3.

33. The method of claim 31, wherein the biological sample is tumour tissue, suspected tumour tissue, a tissue biopsy, mucus, stool, tear, urine, blood, serum, cell extract, biopsy specimens and/or fluid which has been introduced into the body of an individual and subsequently removed

34. The method of claim 31, where said determining the level of Galectin-3 or expression of LGALS3 or level of LGALS3 expression in a subject is determined before the administration to the subject of the effective amount of the composition of claim 2, preferably wherein:
the administration is carried out when any Galectin-3 or any expression of LGALS3 in the biological sample from said subject is determined; and/or
the administration is carried out when the level of Galectin-3 or expression of LGALS3 or level of LGALS3 expression in the biological sample from said subject is found to be overexpressed or higher than a base line or higher than a reference.

35-36. (canceled)

37. A kit consisting of separate packs, packages or amounts of an effective amount of one or more of: a compound of Formula X, a compound of Formula Y, a compound of Formula Z, a composition comprising a compound of Formula X, a composition comprising a compound of Formula Y, a composition comprising a compound of Formula Z a composition comprising two or more of a compound of Formula X, a compound of Formula Y or a compound of Formula Z, and/or pharmaceutically acceptable salts or pharmaceutical formulations of any of those, and optionally with an effective amount of a further active ingredient and/or instructions.

38. (canceled)

39. A pharmaceutical composition comprising a compound of Formula X, or a pharmaceutically acceptable salt thereof, and/or a compound of Formula Y, or a pharmaceutically acceptable salt thereof, and/or a compound of Formula Z, or a pharmaceutically acceptable salt thereof, in combination with an anti-tumour agent and/or anti-metastatic agent and/or chemotherapy agent, and/or radiotherapy agent immunotherapeutic agent.

40. The pharmaceutical composition of claim 39 for use in inhibiting Galectin-3, preferably wherein the composition is for use in the treatment and/or prevention of a disease, disorder, state or condition wherein inhibiting Galectin-3 would be beneficial for the treatment and/or prevention of the disease, disorder, state or condition, preferably wherein the disease, disorder state or condition is cancer, cancer metastasis, fibrosis, inflammation, diabetes and/or one or more other disease, disorder state or condition affected and/or mediated by Galectin-3.

41-46. (canceled)
